(19) United States
(12) Patent Application Publication
(10) Pub. No.: US 2005/0064023 A1
(43) Pub. Date: Mar. 24, 2005

(54) Compound

(76) Inventors: Michael Jorgensen, London (GB); Michael Keller, London (GB); Adrew David Miller, London (GB); Eric Perouzel, London (GB)

Correspondence Address: Scott A. McCollister;Fay Sharpe Fagan Minnich & McKee, 1100 Superior Avenue, 7th Floor, Cleveland, OH, 44114-2518, US

(21) Appl. No.: 10/496,970

(22) PCT Filed: Dec. 4, 2002
(86) PCT No.: PCT/GB02/05471

(30) Foreign Application Priority Data
Dec. 5, 2001 (GB) 0129121.0

(51) Int. Cl. A61K 9/127

(52) U.S. Cl. 424/450

(57) ABSTRACT

The present invention provides a delivery vehicle for a therapeutic agent comprising a modified lipid and a therapeutic agent; wherein the modified lipid comprises a lipid and a delivery, targeting or stabilising moiety (DTS moiety); wherein the lipid is linked to the DTS moiety via a linker which is stable in biological fluid and which is unstable in defined conditions; and wherein the DTS moiety is linked to the lipid alter formation of a complex of lipid and therapeutic agent.

The present invention relates to a compound and a delivery vehicle. In addition, the present invention relates to processes for making the compound and delivery vehicle and to the use of that compound and delivery vehicle in therapy, in particular gene therapy (especially gene transfer) and drug delivery.

One aspect of gene therapy involves the introduction of foreign nucleic acid (such as DNA) into cells, so that its expressed protein may carry out a desired therapeutic function.

Examples of this type of therapy include the insertion of TK, TSG or ILG genes to treat cancer; the insertion of the CFTR gene to treat cystic fibrosis; the insertion of NGF, TH or LDL genes to treat neurodegenerative and cardiovascular disorders; the insertion of the IL-1 antagonist gene to treat rheumatoid arthritis; the insertion of HIV antigens and the TK gene to treat AIDS and CMV infections; the insertion of antigens and cytokines to act as vaccines; and the insertion of β-globin to treat haemoglobinopathic conditions, such as thalassaemias.

Many current gene therapy studies utilise adenoviral gene vectors—such as Ad3 or Ad5- or other gene vectors. However, serious problems have been associated with their use. This has prompted the development of less hazardous, non-viral approaches to gene transfer.

A non-viral transfer system of great potential involves the use of cationic liposomes. In this regard, cationic liposomes—which usually consist of a neutral phospholipid and a cationic lipid—have been used to transfer DNA, mRNA, antisense oligonucleotides, proteins, and drugs into cells. A number of cationic liposomes are commercially available and many new cationic lipids have recently been synthesised. The efficacy of these liposomes has been illustrated by both in vitro and in vivo.

A cytofectin useful in the preparation of a cationic liposome is N-[1-(2,3-dioleoyloxy)propyl]-N,N,N-trimethyl ammonium chloride, otherwise known as “DOTMA”.

One of the most commonly used cationic liposome systems consists of a mixture of a neutral phospholipid dioleoylphosphatidylethanolamine (commonly known as “DOPE”) and a cationic lipid, 3β-[(N,N-dimethylaminoethane)carbamoyl]cholesterol (commonly known as “DC-Chol”).

Despite the efficacy of the known cationic liposomes there is still a need to optimise the gene transfer efficiency of cationic liposomes in human gene therapy. With the completion of the human genome project, the use of genes for therapeutic purposes, described as gene therapy is increasingly expected to revolutionise medicine. In this context, even though still less effective than viral technology, non-viral delivery is increasingly recognised by the scientific community as the safest option for human applications.

This field has evolved considerably in the last decade with the apparition of complex macromolecular constructs including many elements of different existing technologies (viral proteins or peptides, liposomes, polymers, targeting strategies and stealth properties).

WO01/48233 teaches a system based on a triplex composed of a viral core peptide Mu, plasmid DNA and cationic Liposome (LMD). This technology gave us good success in vitro and promising results in vivo. But as for all existing non-viral technology more development is needed to achieve a therapeutic level in vivo.

WO01/48233 and WO02/48380 teaches a system based on modified lipid wherein the lipid carries a carbohydrate moiety. These modified lipids have been found to be stable and have low toxicity.

To this end, formulation must achieve stability of the particle in biological fluids (serum, lung mucus) and still maintain efficient transfection abilities.

This requirement is one of the main hurdles of all existing technology. Current stable formulations achieve little transfection and most present efficient transfecting agents are drastically limited in the scope of their application due to this instability.

After administration (in blood for systemic application or in mucus for lung topical administration), the charged complexes are exposed to salt and biological macromolecules leading to strong colloidal aggregation and adsorption of biological active elements (opsonins) at their surface. The gene vehicles undergo drastic changes that could include precipitation, binding of proteins leading to particle elimination by macrophages and surface perturbation resulting in its destruction.

With the aim of generating drug and gene delivery systems for cell specific targeting in vitro and in vivo, protocols are required for the production of biological fluid-stable delivery systems with sufficient activity to exhibit therapeutic benefits. Therefore, a balance between stability and activity must be found for an efficient drug/gene delivery vehicle.

A handful of approaches described in literature use acid labile lipids which are thought to being cleaved after endocytosis within endosomes and thus to help the release of the drug or pDNA into the cytosol.

Acid Labile or Reduction Sensitive Lipids to Enhance Drug/pDNA Release—there has been taught the following strategies to introduce acid-labile (esters, vinyl ethers) or reduction sensitive-linkers (disulfides) within liposomes/lipoplexes to aid the release of the drug or gene from acidic compartments such as endosomes.

Ortho-esters: Exposure of ortho-ester containing lipids to pH 4.5 resulted in complete hydrolysis of the compound at 38° C. over a non-indicated exposure time according to Nantz et al. (1). However, pH 4.5 is lysosomal conditions, whereas the author's claim of potential endosome escape by liposome formulations containing these novel lipids cannot be justified due to the range of pH (6 in early endosomes, 5 in late endosomes) encountered in endosomal compartments.

Diplasmenyl lipids: Vinyl-ether containing lipids are efficiently hydrolysed to fatty acid aldehydes and glycerophosphocholine, leading to enhanced liposome permeability when >20% of the lipid has been hydrolysed according to Thompson et al. (2, 3). This system maybe interesting for classical drug delivery. However, no data for gene delivery is presented but are announced being in press.

Disulfide bonds: Hughes et al. reported the introduction of disulfide bonds into lipids that selectively destabilize the pDNA/liposome complex in reductive environments such as endosome and cytosol. The lipid, 1,2-dioleyl-sn-glycero-3-succinyl-2-hydroxyethyl-disulfide omithine (DOGSDSO) was used in combination with DOPE (4). Enhancement of up to 50 times compared to the non-disulfide analogs of the lipids were reported (5). In analogy, cholesteryl-hemi-dithio-diglycolyl-tris(aminoethyl)-amine (CHDTAEA) was prepared and used in combination with DOPE as neutral helper lipid (6). Both an increase of transfection efficiency and a decrease of cellular toxicity were observed when compared to DC-Chol liposomes. Reduction-sensitive lipopolyamines (RSLs) as a novel non-viral gene delivery system for modulated release of pDNA with improved transgene expression were described by Scherman et al. (7). These compounds harbour disulfide bridges within different positions in the backbone of the lipids, form micelles and compact pDNA to small particles of about 100 nm diameter. They were reported to be sensitive to reductive conditions and serum.

PEG lipids with disulfide bonds: A new detachable polyethylene glycol conjugate mPEG-DTB-DSPE which regenerates natural phospholipid DSPE upon exposure to reducing conditions was reported by Huang et al. (8). A formulation of DOPE:mPEG-DTB-DSPE (100:3, m/m) appears to release an entrapped fluorophore within 30 minutes at pH 7.2, 37° C. in the presence of 1 mM Cys.

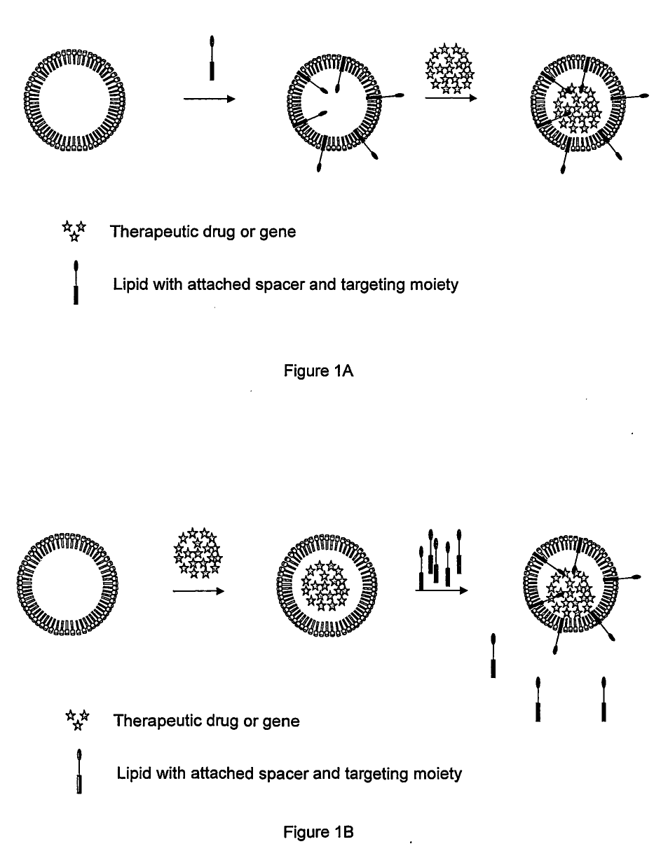

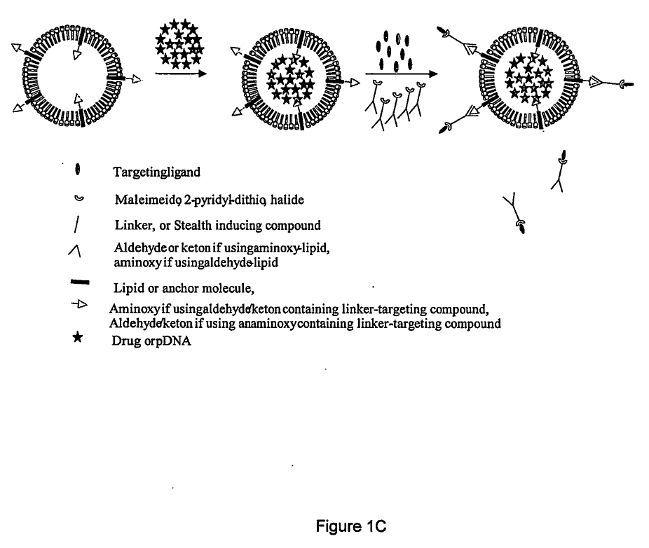

Production of Stealth Liposomes by Post-Coating (Conjugation)—Most current protocols for the production of stealth liposomes or lipoplexes use polyethylene glycol-linked lipids that are pre-incubated into the delivery vector (FIGS. 1A and 1B). Only recently, post-coated strategies were reported by Wagner (9) and by Xu (10) using established thiol chemistry or amide bond chemistry to form a covalent, non hydrolysable linkages with the surface of the liposome/lipoplex.

The present invention alleviates the problems of the prior art.

According to one aspect of the present invention there is provided a delivery vehicle for a therapeutic agent comprising a modified lipid and a therapeutic agent; wherein the modified lipid comprises a lipid and a delivery, targeting or stabilising moiety (DTS moiety); wherein the lipid is linked to the DTS moiety via a linker which is stable in biological fluid and which is unstable in defined conditions; and wherein the DTS moiety is linked to the lipid after formation of a complex of lipid and therapeutic agent.

According to one aspect of the present invention there is provided a process for the preparation of delivery vehicle for a therapeutic agent comprising a modified lipid and a therapeutic agent, the process comprising the steps of; (a) forming a complex of a lipid comprising a linker moiety and the therapeutic agent; (b) linking a delivery, targeting or stabilising moiety (DTS moiety) to the lipid via the linker moiety, wherein the link between the DTS moiety and the lipid is stable in biological fluid and is unstable in defined conditions.

According to one aspect of the present invention there is provided a modified lipid of the formula 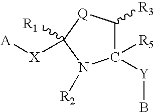
wherein one of A and B is a lipid and the other of A and B is a delivery, targeting or stabilising moiety (DTS moiety); wherein X and Y are independently optional linker groups; wherein R₁ is H or a hydrocarbyl group; wherein R₂ is a lone pair or R₄, wherein R₄ is a suitable substituent; wherein R₃ and R₅ are independently selected from H and a hydrocarbyl group; and wherein Q is selected from O, S, NH

According to one aspect of the present invention there is provided a modified lipid is of the formula 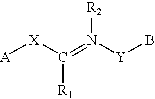
wherein one of A and B is a lipid and the other of A and B is a delivery, targeting or stabilising moiety (DTS moiety); wherein X and Y are independently optional linker groups; wherein R₁ is H, O⁻ or a hydrocarbyl group; and wherein R₂ is a lone pair or R₄, wherein R₄ is a suitable substituent.

According to one aspect of the present invention there is provided a modified lipid is of the formula 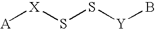
wherein one of A and B is a lipid and the other of A and B is a delivery, targeting or stabilising moiety (DTS moiety); wherein X and Y are independently optional linker groups.

According to another aspect of the present invention there is provided a compound or delivery vehicle according to the present invention or a compound when prepared by the process of the present invention for use in therapy.

According to another aspect of the present invention there is provided the use of a compound or delivery vehicle according to the present invention or a compound or delivery vehicle when prepared by the process of the present invention in the manufacture of a medicament for the treatment of a genetic disorder or a condition or a disease.

According to another aspect of the present invention there is provided a liposome/lipoplex formed from the compound or delivery vehicle according to the present invention or a compound or delivery vehicle when prepared by the process of the present invention.

According to another aspect of the present invention there is provided a method of preparing a liposome/lipoplex comprising forming the liposome/lipoplex from the compound or delivery vehicle according to the present invention or a compound or delivery vehicle when prepared by the process of the present invention.

According to another aspect of the present invention there is provided a liposome/lipoplex according to the present invention or a liposome/lipoplex as prepared by the method of the present invention for use in therapy.

According to another aspect of the present invention there is provided the use of a liposome/lipoplex according to the present invention or a liposome/lipoplex as prepared by the method of the present invention in the manufacture of a medicament for the treatment of genetic disorder or condition or disease.

According to another aspect of the present invention there is provided a combination of a nucleotide sequence or a pharmaceutically active agent and any one or more of: a compound or delivery vehicle according to the present invention, a compound or delivery vehicle when prepared by the process of the present invention, a liposome/lipoplex of the present invention, or a liposome/lipoplex as prepared by the method of the present invention.

According to another aspect of the present invention there is provided a combination according to the present invention for use in therapy.

According to another aspect of the present invention there is provided the use of a combination according to the present invention in the manufacture of a medicament for the treatment of genetic disorder or condition or disease.

According to another aspect of the present invention there is provided a pharmaceutical composition comprising a compound or delivery vehicle according to the present invention or a compound or delivery vehicle when prepared by the process of the present invention admixed with a pharmaceutical and, optionally, admixed with a pharmaceutically acceptable diluent, carrier or excipient.

According to another aspect of the present invention there is provided a pharmaceutical composition comprising a liposome/lipoplex according to the present invention or a liposome/lipoplex as prepared by the method of the present invention admixed with a pharmaceutical and, optionally, admixed with a pharmaceutically acceptable diluent, carrier or excipient.

Some further aspects of the invention are defined in the appended claims.

We have found the provision of particular delivery vehicles containing therapeutic agents such as nucleotides or other pharmaceutically active agents such as “small molecules” are advantageous in a number of respects. Provision of a delivery vehicle comprising a lipid and a DTS moiety wherein the link between them is stable in extracellular biological fluid and which is unstable in intracellular biological fluid and/or defined conditions; allows for
surface protection and/or functionalisation (targeting) of drug and gene delivery systems without compromising the core vector integrity.
temporary or permanent introduction of DTS moieties such as targeting moieties to drug or gene delivery vehicles. The permanence of the DTS moiety may be controlled according to the choice of groups on either the lipid or the DTS moiety.
One pot reaction affording the self-assembly of drug/gene delivery vehicle with DTS plus target molecule. The self-assembly may comprise a single assembly or comprise a staged assembly provided by staged reactions in a single pot. In either aspect this methodology avoids extensive purification procedures by simple dialysis of excess, non-reacted reagents.
The strength of attachment of the DTS moiety to the lipid is triggerable to undergo hydrolysis in specific pH conditions.

The post-coating one-pot methodology of the present process is typically based on selective and high reactivity of an aminoxy to react with aldehydes and ketones to form —C═N-(Schiff-base like) covalent linkages. Importantly, the reaction can be carried out in aqueous environment at basic or acidic pH. Furthermore, there is no partial breakdown of the reactive group when exposed to aqueous conditions as it is the case for NHS-activated carboxyls and other esters. Therefore, the stability of the reactive species, e.g. the aldehyde/ketone and the aminoxy or thiol and alcohol allows total control of the surface reaction without loss of reactive species due to hydrolysis/degradation. In other words, the number of post-coated compounds (ligands) is easily controlled by the stoichiometry applied of both the ligand (post-conjugated species) and the ligate (reactive species on the surface of liposome/lipoplex/micelles). In addition, the differential reactivity of aldehydes and ketones allows for tuneable stability of the conjugated ligand and ligate. Aldehydes are far more reactive than ketones, thus forming a faster and more stable adduct than the ketone analogues. As a result, aldehydes shall preferably be used to form more stable adducts, whereas ketones will be used to form more labile conjugations. However, it has to be emphasised that, depending on the nature of substituents, both aldehydes and ketones can exhibit differential stability with the aminoxy-containing compound. Therefore, both aldehydes and ketones can be applied for temporary and permanent linkages.

We have achieved post-couplings of a stabilising moieties, such as polyethylene-glycol molecules (PEG), using chemoselective acid-labile and non-labile coupling strategies to lipoplexes. This affords substantial increase in the resistance to serum-induced degradation and precipitation of lipoplexes as well as tuneable release of the stabilising moiety in biologically relevant conditions. The needed degree of stabilisation of the lipoplexes is chosen according to the molar ratio of stabilising moiety applied. We have also found that targeting ability and enhanced transfection efficacy by using targeting moieties, such as Folate post-coupling to PEG-stabilised lipoplexes, may be achieved. This technology also allows simple purification via dialysis.

Acid lability of stabilising moieties, such as the PEG moiety, may be achieved when Schiff-bases are formed between the stabilising moiety and the lipoplex, for example between the PEG and amines or hydrazyde units. Acid resistance may be achieved by reacting the stabilising moiety to aminoxy units of the lipoplex. A particularly promising stabilising unit is dialdehyde-PEG, which can be used to stabilise lipoplexes through formation of a Schiff-base with one aldehyde. The second aldehyde can be used for targeting purpose by adding an aminoxy-containing targeting ligand.

Preferred Aspects

In one preferred aspect the link is unstable on contact with a cell surface or within a cell.

In one preferred aspect the link is unstable at defined pH conditions. One skilled in the art would be able to engineer the link so as to be unstable at required pH conditions. The required pH condition will typically be those which differ significantly from those in which the delivery vehicle or lipid would be found.

In one preferred aspect the link is unstable at a pH of from 5 to 6.5 such 5.3 to 6.2, or 5 to 6 or 5.5. to 6.5. Other pHs may be envisaged by one skilled in the art. For example, the link may be unstable at a pH found in a tumour cell, this is typically from 6.5 to 7.0. The link may be unstable at a pH found in a gastrointestinal tract, for example in a stomach which typically is at a pH of from 1.5 to 2.5.

In one preferred aspect the link is unstable under reductive conditions.

It will be appreciated by one skilled in the art that any suitable linker may be provided which is stable in biological fluid and which is unstable in defined conditions. Preferred linkers are described herein.

In one preferred aspect the modified lipid of the formula 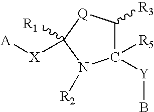
wherein one of A and B is a lipid and the other of A and B is a delivery, targeting or stabilising moiety (DTS moiety); wherein X and Y are independently optional linker groups; wherein R₁ is H or a hydrocarbyl group; wherein R₂ is a lone pair or R₄, wherein R₄ is a suitable substituent; wherein R₃ and R₅ are independently selected from H and a hydrocarbyl group; and wherein Q is selected from O, S, NH.

The term “hydrocarbyl group” as used herein means a group comprising at least C and H and may optionally comprise one or more other suitable substituents. Examples of such substituents may include halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In one preferred aspect the modified lipid is of the formula 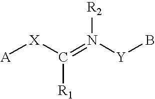
wherein one of A and B is a lipid and the other of A and B is a delivery, targeting or stabilising moiety (DTS moiety); wherein X and Y are independently optional linker groups; wherein R₁ is H, O⁻ or a hydrocarbyl group; and wherein R₂ is a lone pair or R₄, wherein R₄ is a suitable substituent.

In one preferred aspect the modified lipid is of the formula 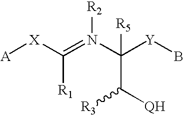
wherein one of A and B is a lipid and the other of A and B is a delivery, targeting or stabilising moiety (DTS moiety); wherein X and Y are independently optional linker groups; and wherein R₁ is H, O⁻ or a hydrocarbyl group; wherein R₂ is a lone pair or R₄, wherein R₄ is a suitable substituent; wherein R₃ and R₅ are independently selected from H and a hydrocarbyl group; and Q is a suitable substituent.

Preferably R₂ is R₄. R₄ may be selected from any suitable substituent. Suitable substituents include electron withdrawing groups such as halogenated hydrocarbons, in particular fluorinated hydrocarbons, nitrophenol such as para-nitro phenol.

Preferably Q is selected from OH, SH, primary amines, secondary amines, tertiary amines and hydrocarbyl.

In one preferred aspect the modified lipid is of the formula 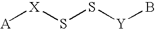
wherein one of A and B is a lipid and the other of A and B is a delivery, targeting or stabilising moiety (DTS moiety); wherein X and Y are independently optional linker groups.

In a preferred aspect A is a DTS moiety and B is a lipid. It will be appreciated by one skilled in the art that A may be a lipid and B may be DTS moiety.

Optional Linker Y

In a preferred aspect optional linker Y is present.

Y may be selected in one aspect from O, S, NH and a hydrocarbyl group.

In one a preferred aspect Y is O (oxygen). In this aspect the modified lipid of the present invention may be of the formula

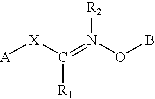

In one alternative aspect Y is a hydrocarbyl group.

Preferably Y is selected from —[CₙHₙ₋₂]ₐ—[NH]_{b}—[CZ]_{c}—[NH]_{d}—[CZ]ₑ—NH—, wherein a, b, c, d and e are independently selected from 0 to 10; wherein n is from 5 to 10; and wherein Z is O or S

Preferably a, b, c, d and e are independently selected from 0 to 5, more preferably 0 to 3 or 0, 1 or 2.

In a highly preferred aspect
a is 0 or 1; and/or
b is 0 or 1; and/or
c is 0 or 1; and/or
d is 0, 1 or 2; and/or
e is 0 or 1.

In further highly preferred aspects Z is O.

In further highly preferred aspects n is 5.

In one aspect Y is an oligomeric or polymeric moiety, for example PEG.

In one aspect Y is selected from —NH—, —NH—CO—NH—, —NH—CS—NH—, —NH—CO—NH—NH—CO—NH—, —CO—NH—, and —C₅H₃—NH—.

In one aspect Y is selected from
—NH—(CH₂)₂—NH—C(O)—CH(CH₂OH)—
—NH—(CH₂)₂—NH—C(O)—CH(CH₂SH)—
—NH—(CH₂)₂—NH—C(O)—CH₂O—
—NH—(CH₂)₂—NH—(CH₂)₃—NH—C(O)—CH(CH₂OH)—
—NH—(CH₂)₂—NH—(CH₂)₃—NH—C(O)—CH(CH₂SH)—
—NH—(CH₂)₂—NH—(CH₂)₃—NH—C(O)—CH₂O—,
—NH—CH₂—C(O)—NH—
—NH—.

In a preferred aspect the linker X comprises or is linked to the lipid via a polyamine group.

It is believed that the polyamine group is advantageous because it increases the DNA binding ability and efficiency of gene transfer of the resultant liposome/lipoplex.

In one embodiment, preferably the polyamine group is a unnaturally occurring polyamine. It is believed that the polyamine head-group is advantageous because the increased amino functionality increases the overall positive charge of the liposome/lipoplex. In addition, polyamines are known to both strongly bind and stabilise DNA. In addition, polyamines occur naturally in cells and so it is believed that toxicological problems are minimised.

In another embodiment, preferably two or more of the amine groups of the polyamine group of the present invention are separated by one or more groups which are not found in nature that separate amine groups of naturally occurring polyamine compounds (i.e. preferably the polyamine group of the present invention has un-natural spacing).

Preferably the polyamine group contains at least two amines of the polyamine group that are separated (spaced from each other) from each other by an ethylene (—CH₂CH₂—) group.

Preferably each of the amines of the polyamine group are separated (spaced from each other) by an ethylene (—CH₂CH₂—) group.

Typical examples of suitable polyamines include spermidine, spermine, caldopentamine, norspermidine and norspermine. Preferably the polyamine is spermidine or spermine as these polyamines are known to interact with single or double stranded DNA. An alternative preferred polyamine is caldopentamine.

Optional Linker X

In a preferred aspect optional linker X is present.

In one aspect optional linker X is not present.

In a preferred aspect X is a hydrocarbyl group.

In one aspect, if X is present is a hydrocarbon group. It may be a hydrocarbon group selected from optionally substituted alkyl groups, alkenyl groups, and alkynyl groups. It may be a hydrocarbon group selected from optionally substituted alkyl groups, alkenyl groups, and alkynyl groups and having from 1 to 10 carbons.

R₁

As discussed above the permanence of the DTS moiety may be controlled according to the choice of R₁ group (in the process or composition of the present invention—the choice of aldehyde or ketone) on either the lipid or the DTS moiety.

In a preferred aspect R₁ is selected from H and hydrocarbyl groups.

In a preferred aspect R₁ is selected from H and hydrocarbon groups.

In a preferred aspect R₁ is selected from H and hydrocarbon groups having from 1 to 10 carbon atoms.

In a preferred aspect R₁ is selected from H, alkyl groups having from 1 to 10 carbon atoms and aryl groups having from 1 to 10 carbon atoms.

In a preferred aspect R₁ is selected from H, alkyl groups having from 1 to 5 carbon atoms (such as methyl and ethyl groups) and aryl groups having 6 carbon atoms.

In a preferred aspect R₁ is H

R₂

Preferably R₂ is R₄. R₄ may be selected from any suitable substituent. Suitable substituents include electron withdrawing groups such as halogenated hydrocarbons, in particular fluorinated hydrocarbons, nitrophenol such as para-nitro phenol.

In one aspect, R₄ is selected from H, and optionally substituted alkyl groups, alkenyl groups, and alkynyl groups. R₄ may be selected from H, and optionally substituted alkyl groups, alkenyl groups, and alkynyl groups and having from 1 to 10 carbons.

In one aspect R₄ is H.

In one aspect R₂ is H.

C═N

The C═N bond may be acid labile or acid resistant.

In a preferred aspect the C═N bond is acid labile.

In one aspect the C═N bond is acid resistant.

DTS Moiety

The delivery, targeting or stabilising moiety (DTS moiety) is provided to enhance the biological properties of the lipid, for example by improving its stability, solubility, bioavailibity and/or affinity for particular biological material (targeting)

In one preferred aspect the DTS moiety is a delivery and/or stabilising moiety.

In one preferred aspect the DTS moiety is a delivery and/or stabilising polymer.

Preferably the DTS moiety is selected from mono or bifunctional poly(ethyleneglycol) (“PEG”), poly(vinyl alcohol) (“PVA”); other poly(alkylene oxides) such as poly(propylene glycol) (“PPG”); and poly(oxyethylated polyols) such as poly(oxyethylated glycerol), poly(oxyethylated sorbitol), and poly(oxyethylated glucose), and the like.

As discussed in background teaching US-A-2001/0021763, the polymers can be homopolymers or random or block copolymers and terpolymers based on the monomers of the above polymers, straight chain or branched, or substituted or unsubstituted similar to mPEG and other capped, monofunctional PEGs having a single active site available for attachment to a linker.

Specific examples of suitable additional polymers include poly(oxazoline), poly(acryloylmorpholine) (“PAcM”), and poly(vinylpyrrolidone)(“PVP”). PVP and poly(oxazoline) are well known polymers in the art and their preparation and use in the syntheses described for mPEG should be readily apparent to the skilled artisan. PAcM and its synthesis and use are described in U.S. Pat. No. 5,629,384 and U.S. Pat. No. 5,631,322.

Suitable targeting moieties which may be utilised in the present invention include antibodies, for example humanized monoclonal antibodies (Her_neu) and single chain human antibody fragments (e.g. Fv), ligands such as folate moieties, carbohydrate epitopes (GM3, aminolactose, vitamins, growth factors, peptides, for example RGD and tenascin, and proteins such as transferin and albumin.

Suitable delivery moieties which may be utilised in the present invention include membrane active peptides and proteins, for example toxins and TAT.

In a preferred aspect of the present invention the DTS moiety comprises a further linker group which is capable of linking to a further moiety such as a DTS moiety. Thus one may provide a DTS which can be further modified to include an additional DTS moiety to modify the functionality of the compound. For example the first DTS moiety may stabilise a liposome/lipoplex formed by the lipid to which the DTS is attached. After formation of the liposome/lipoplex a further DTS may be provided which is useful in targeting the liposome/lipoplex to a specific biological target.

The further linker may be selected from a maleimeido group, halogenated carbon, aldehydes and ketones. The further linker is preferably a ketone.

The further linker may be provided by initially linking to a first DTS moiety which has at least two groups capable of forming links. A first of the two groups may be utilised in linking the first DTS moiety to the lipid. The second of the two groups may be utilised in linking a second DTS moiety to the initial DTS/lipid complex. Preferably the first DTS moiety is a stabilising moiety. In this aspect the system is stabilised prior to further modification. Preferably the second DTS moiety is a targeting moiety.

Lipid

In a preferred aspect the lipid is or comprises a cholesterol group or a glycerol/ceramide backbone. Any lipid-like structure or polyamine is suitable.

Preferably the cholesterol group is cholesterol.

Preferably the cholesterol group is linked to X or Y via a carbamoyl linkage.

The cholesterol group can be cholesterol or a derivative thereof. Examples of cholesterol derivatives include substituted derivatives wherein one or more of the cyclic CH₂ or CH groups and/or one or more of the straight-chain CH₂ or CH groups is/are appropriately substituted. Alternatively, or in addition, one or more of the cyclic groups and/or one or more of the straight-chain groups may be unsaturated.

In a preferred embodiment the cholesterol group is cholesterol. It is believed that cholesterol is advantageous as it stabilises the resultant liposomal bilayer.

Preferably the cholesterol group is linked to the optional linker group via a carbamoyl linkage. It is believed that this linkage is advantageous as the resultant liposome/lipoplex has a low or minimal cytotoxicity.

In a highly preferred aspect the lipid is —C(═O)—O-Chol. In a further highly preferred aspect B is the lipid —C(═O)—O-Chol.

Further Aspects

The modified lipid of the present invention of the formula 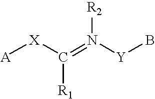
may be prepared by any process. We have found that production from an aminoxy compound and an aldehyde or ketone is particularly advantageous.

According to another aspect of the present invention there is provided a process for preparing a modified lipid of the formula 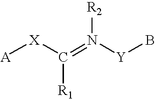
comprising reacting (i) a compound of the formula; and 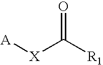
(ii) a compound of the formula 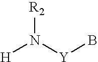
wherein one of A or B is a lipid and the other of A and B is a delivery, targeting or stabilising moiety (DTS moiety); wherein X and Y are independently optional linker groups; wherein R₁ is H, O⁻ or a hydrocarbyl group and wherein R₂ is a lone pair, H, hydrocarbyl group.

According to another aspect of the present invention there is provided a composition comprising (i) a compound of the formula 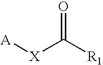
(ii) a compound of the formula 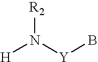
wherein one of A or B is a lipid and the other of A and B is a delivery, targeting or stabilising moiety (DTS moiety); wherein X and Y are independently optional linker groups; wherein R₁ and R₂ are independently H or a hydrocarbyl group.

Preferably R₂ is H or a hydrocarbyl group.

In a preferred aspect R₂ is H.

Preferably the process of the present invention is an aqueous medium or in a wholly aqueous medium.

The present invention further provides a compound prepared by a process of the present invention defined herein, a compound obtained by a process of the present invention defined herein, and/or a compound obtainable by a process of the present invention defined herein.

Preferably the compound is in admixture with or associated with a nucleotide sequence.

The nucleotide sequence may be part or all of an expression system that may be useful in therapy, such as gene therapy.

In a preferred aspect the compound of the present invention is in admixture with a condensed polypeptide/nucleic acid complex to provide a non-viral nucleic acid delivery vector. The condensed polypeptide/nucleic acid complex preferably include those disclosed in our copending application WO01/48233. Preferably the polypeptides or derivatives thereof are capable of binding to the nucleic acid complex. Preferably the polypeptides or derivatives thereof are capable of condensing the nucleic acid complex. Preferably the nucleic acid complex is heterologous to the polypeptides or derivatives thereof.

Preferably the compound is in admixture with or associated with a pharmaceutically active agent. The pharmaceutically active agent may be selected from PNA, ODN, RNA, DNA, peptides, proteins and drugs such as the anticancer drug doxorubicin.

Preferably, the cationic liposome/lipoplex is formed from the compound of the present invention and a neutral phospholipid—such as DOTMA or DOPE. Preferably, the neutral phospholipid is DOPE.

The present invention will now be described in further detail by way of example only with reference to the accompanying figures in which:

FIG. 1A shows pre-insertion of targeting moieties into liposomes post-loading;

FIG. 1B shows post-insertion of targeting moieties into liposomes;

FIG. 1C shows one pot coupling of spacer, targeting compound in aqueous environment to preloaded drug/gene carrier system

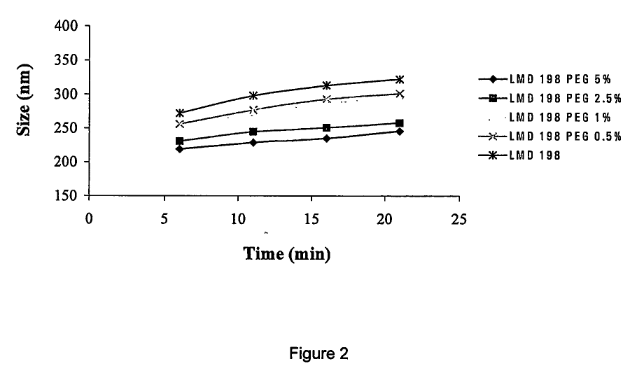

FIG. 2 shows stability Assays of LMD(B198) in OptiMEM in Presence of PEG-bis-CHO

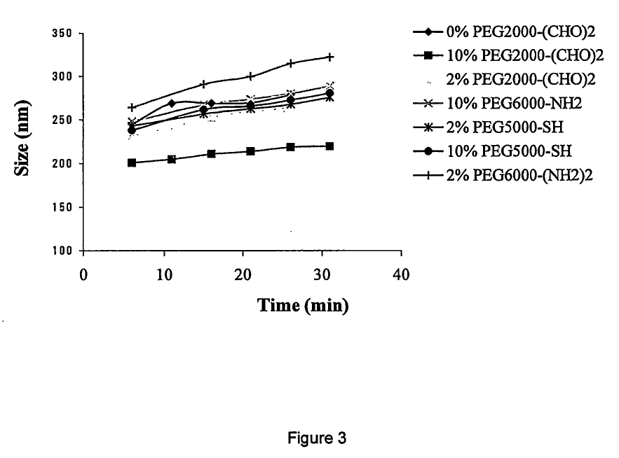

FIG. 3 shows Stability Assays of LMD(B198/DOPE) (40:60) in OptiMEM

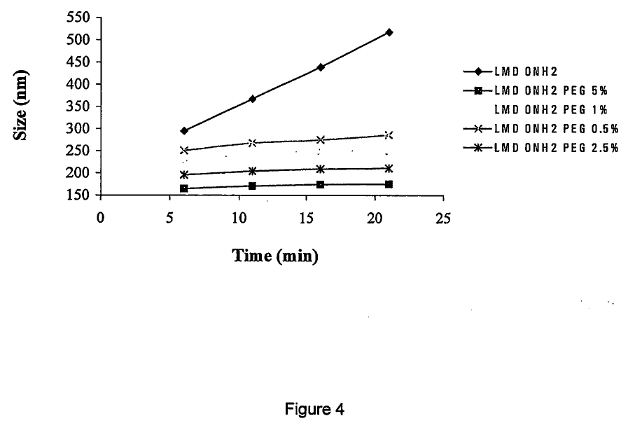

FIG. 4 shows Stability of LMD(B198/aminoxylipid 1) (30:10) in presence of PEG-bis-CHO in OptiMEM

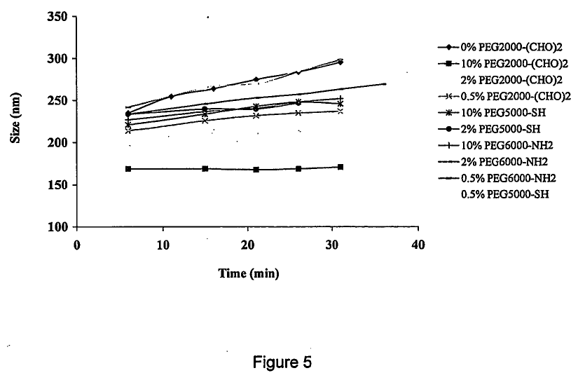

FIG. 5 shows Stability Assays of LMD(B198/aminoxylipid 1/DOPE) (30:10:60) in OptiMEM

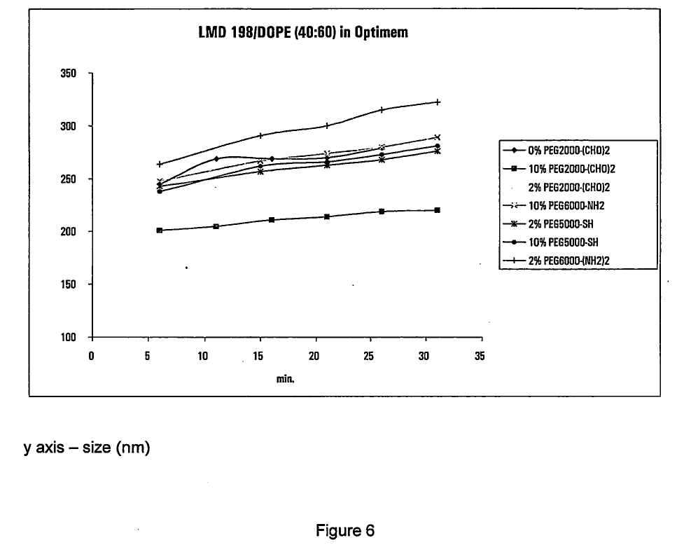

FIG. 6 shows a graph

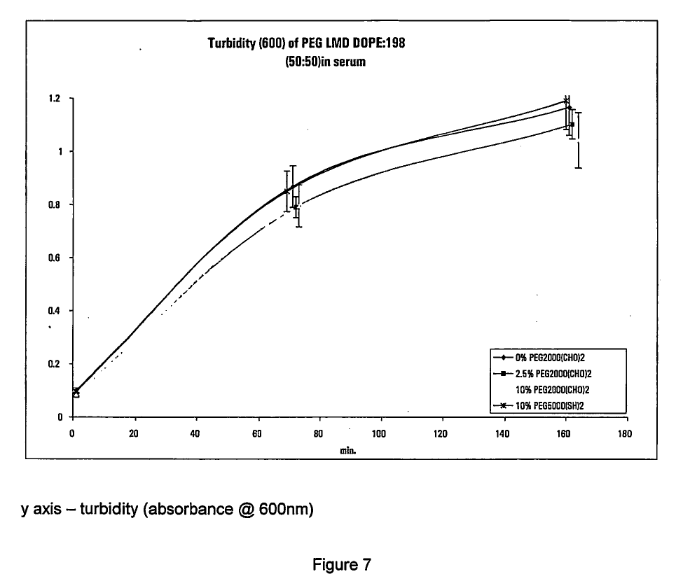

FIG. 7 shows a graph

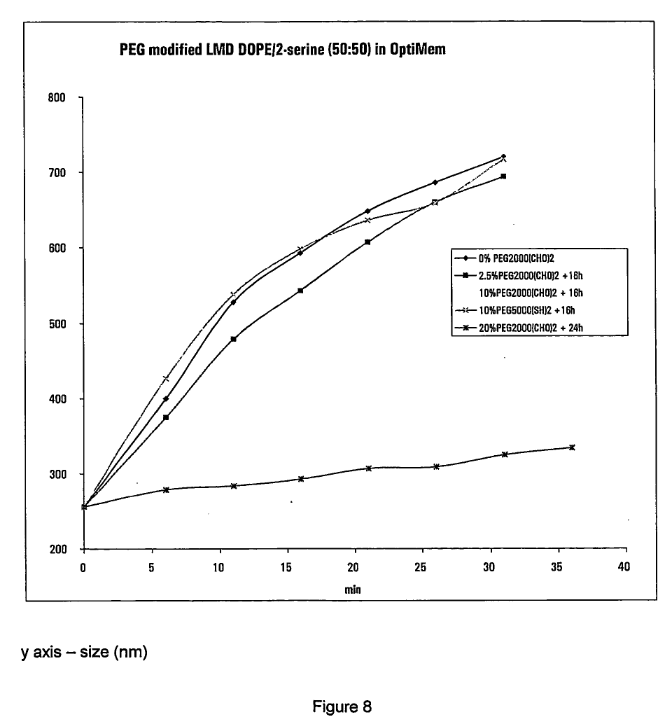

FIG. 8 shows a graph

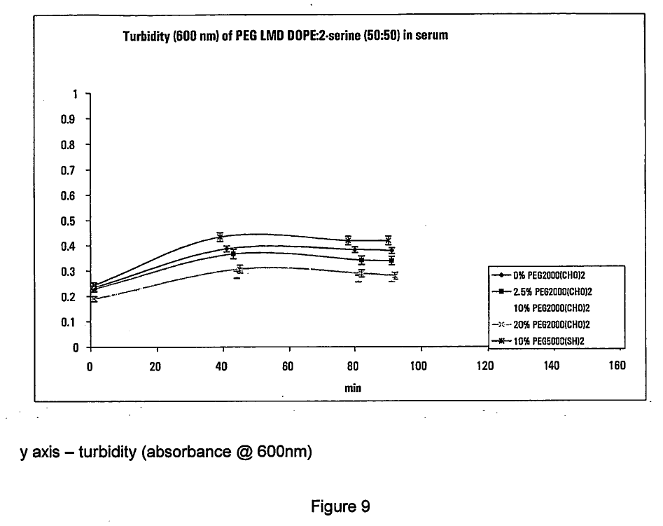

FIG. 9 shows a graph

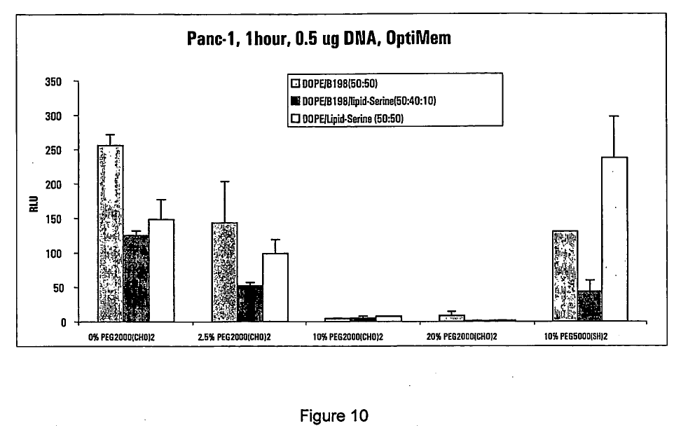

FIG. 10 shows a graph

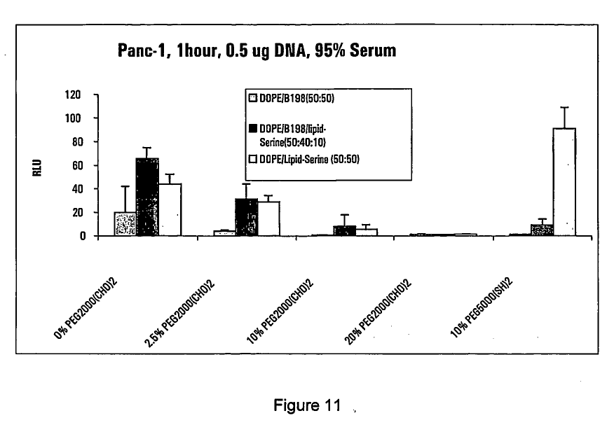

FIG. 11 shows a graph

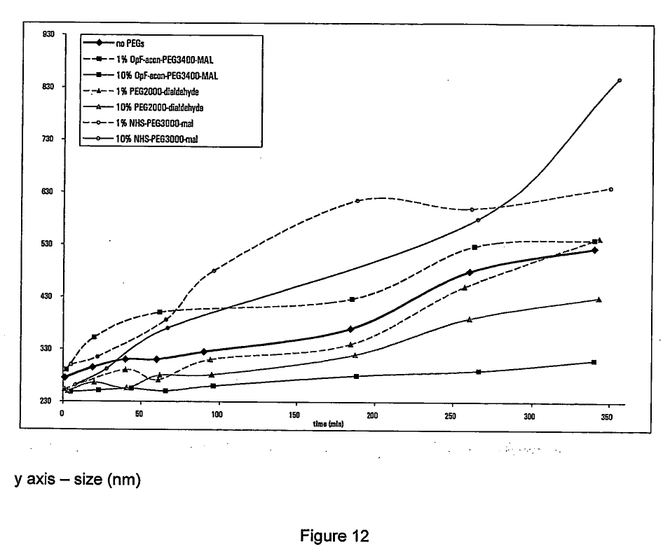

FIG. 12 shows size measurement of LD DOPE:lipidB198:cholesterol (45:30:25, m/m/m) modified with different PEGs after incubation in serum.

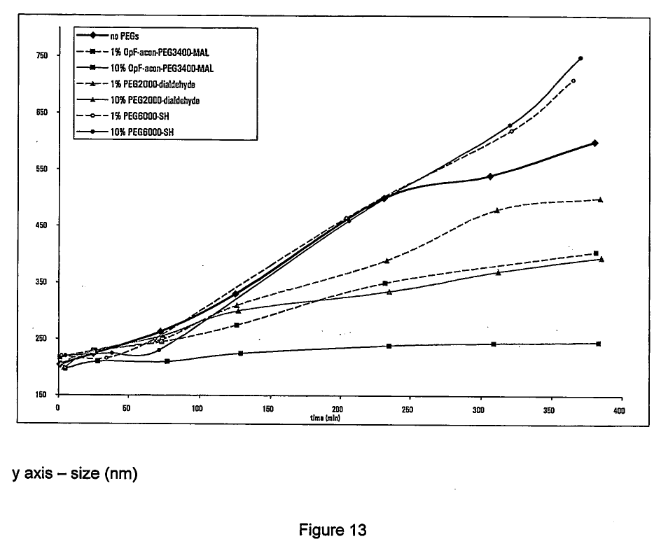

FIG. 13 shows size profiles of LD (DOPE:lipidB198):lipid 23 (45:30:25, m/m/m) modified with different PEGs after incubation in serum measured by PCS.

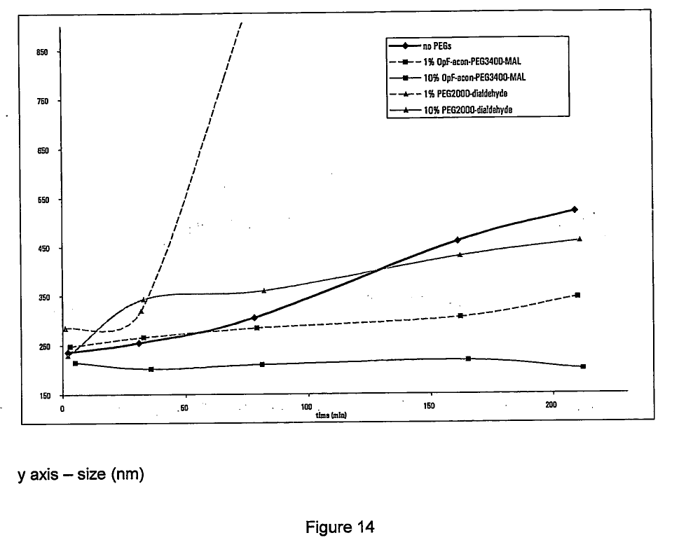

FIG. 14 shows size measurement of LD DOPE:lipidB198:lipid 23 (45:30:25, molar ratios) modified with different PEGs after 3 h incubation at pH 5.3 followed by serum addition.

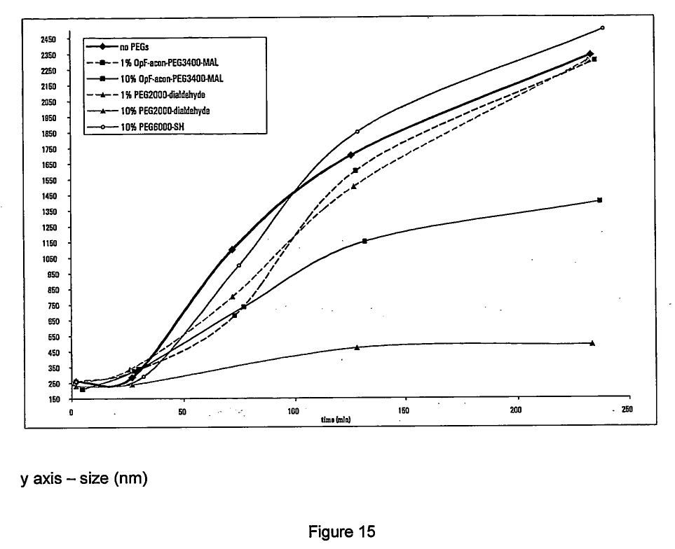

FIG. 15 shows size measurement of LD DOPE:lipidB198:aminoxy-lipid-1 (45:30:25, m/m/m) modified with different molar % of PEGs after incubation in serum.

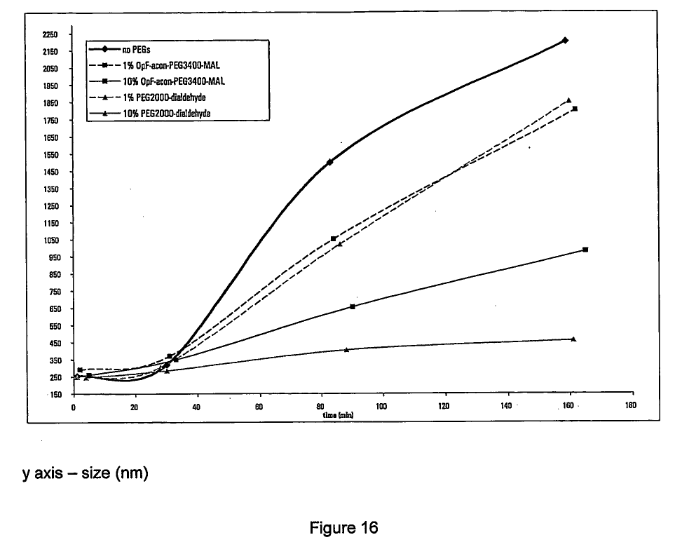

FIG. 16 shows size measurement of LD DOPE:lipidB198:lipid-aminoxy 1 (45:30:25, molar ratios) modified with different molar % of PEGs after 3 h incubation at pH 5.3 followed by serum addition.

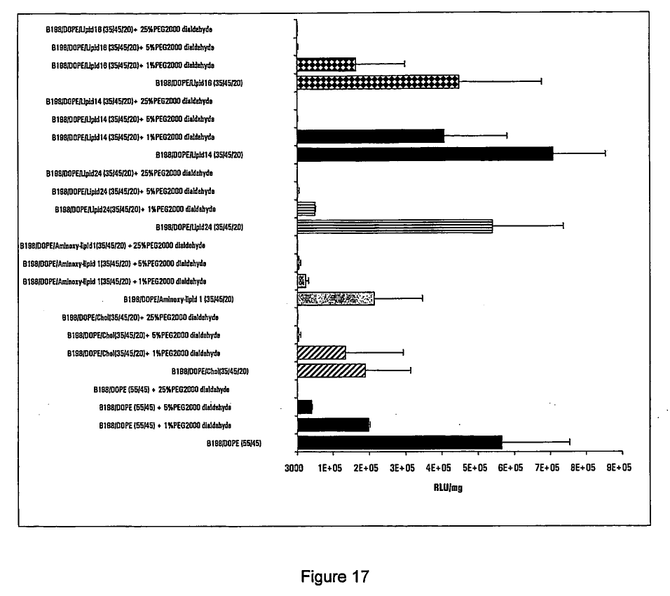

FIG. 17 shows transfection of various LDs modified with different molar percentages of PEG²⁰⁰⁰-dialdehyde in Panc-1 cells.

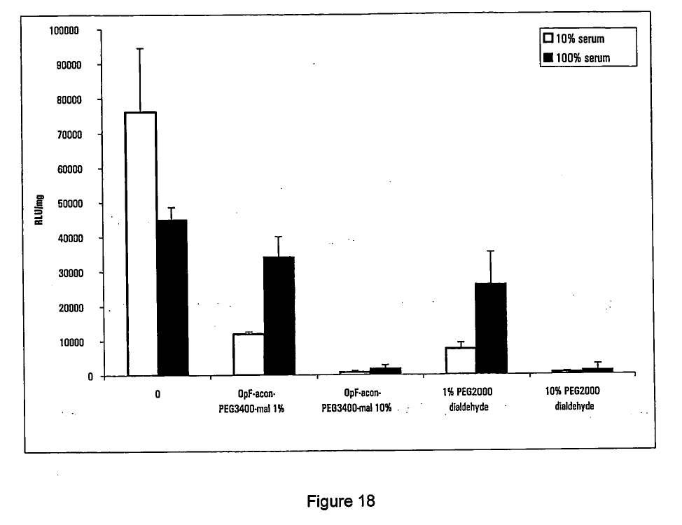

FIG. 18 shows LD composed of DOPE:lipidB198:lipid-aminoxylipid 1 (45:30:25, molar ratios) liposomes (ratio pDNA:lipid=1:12) at 0.1 mg/ml (pDNA) were modified with different PEGs at 1 and 10 molar percentage and transfected on OVCAR-1 cells.

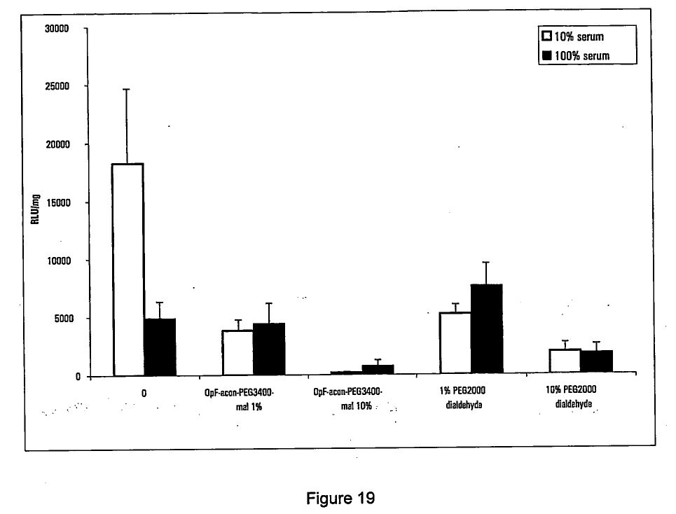

FIG. 19 shows LD composed of DOPE:lipidB198:lipid 23 (45:30:25, m/m/m) liposomes (ratio pDNA:lipid=1:13) at 0.1 mg/ml (pDNA) were modified with different PEGs at 1 and 10 molar percentage and transfected on OVCAR-1 cells. 0 corresponds to no PEG.

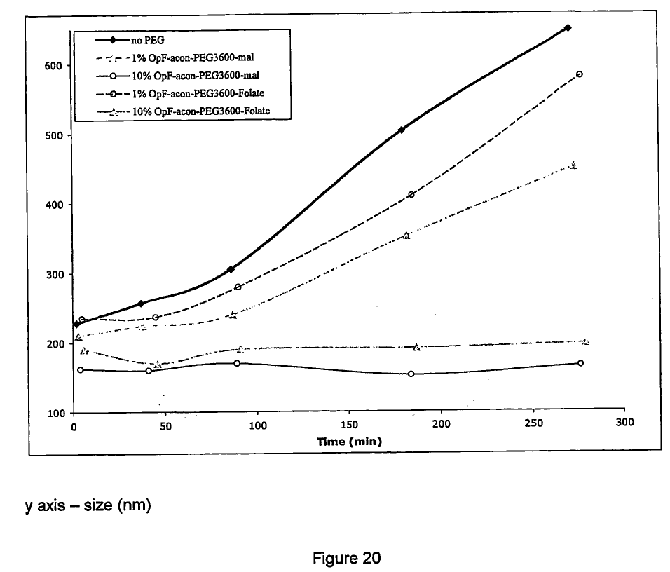

FIG. 20 shows size measurement of LD DOPE:lipidB198:lipid 23 (45:30:25, m/m/m) modified with different molar % of PEGs after incubation in serum.

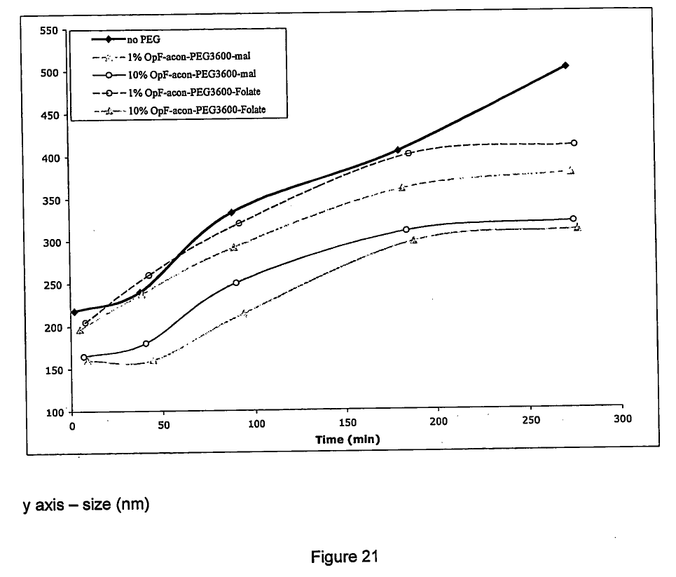

FIG. 21 shows size measurement of LD DOPE:lipidB198:aminoxy-lipid-1 (45:30:25, m/m/m) modified with different molar % of PEGs after incubation in serum.

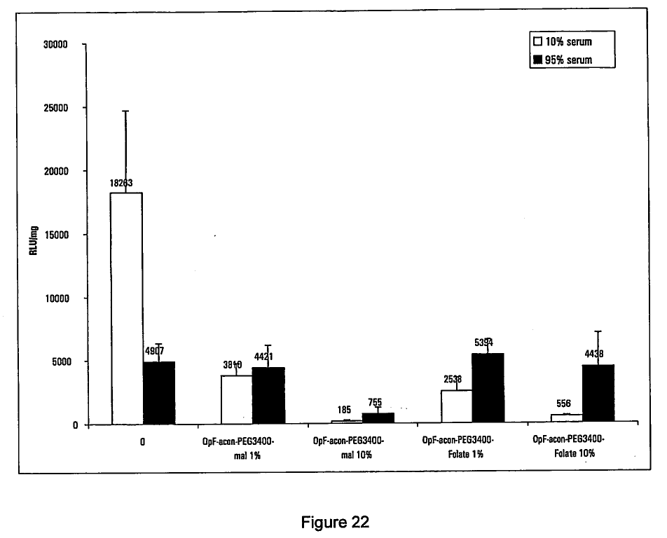

FIG. 22 shows LD composed of DOPE:LipidB198:lipid 23 (45:30:25, m/m/m) liposomes (ratio pDNA:lipid=1:14) at 0.1 mg/ml (pDNA) were subjected to targeting experiments and transfected on OVCAR-1 cells.

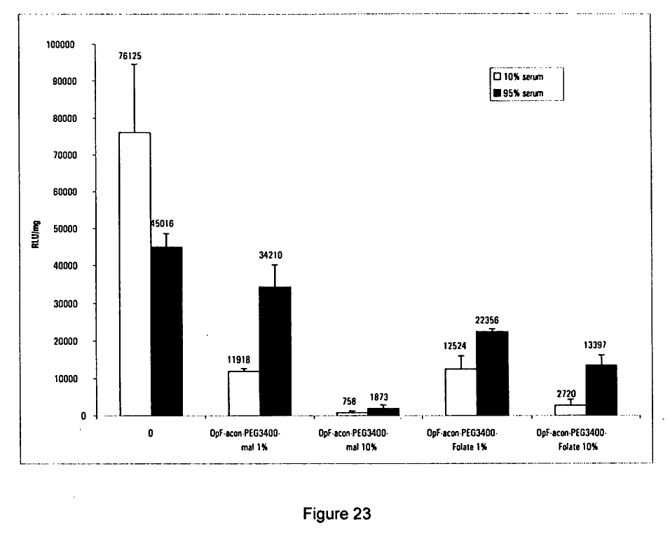

FIG. 23 shows LD composed of DOPE:LipidB198: lipid-aminoxylipid 1 (45:30:25, m/m/m) liposomes (ratio pDNA:lipid=1:12) at 0.1 mg/ml (pDNA) were subjected to targeting experiments and transfected on OVCAR-1 cells.

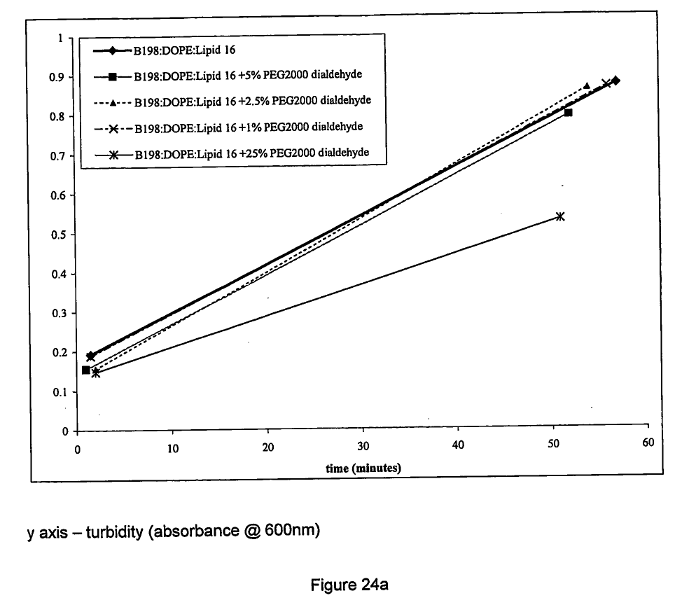

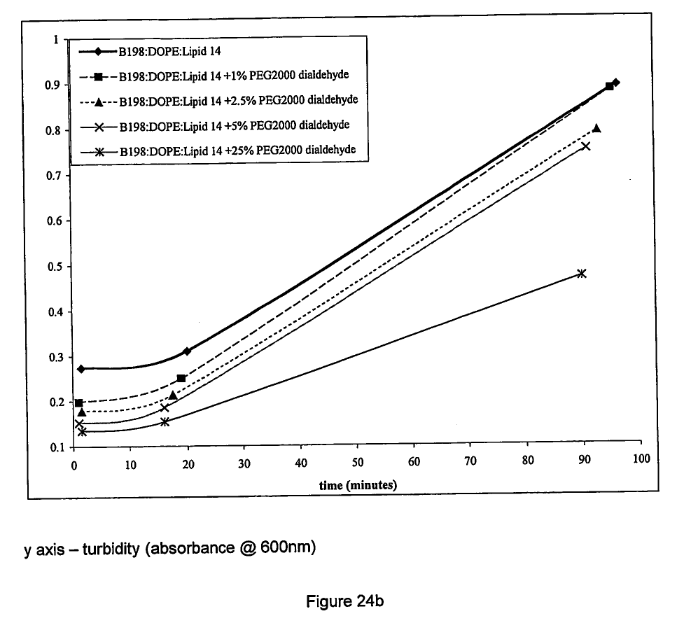

FIG. 24a shows turbidity measurement of LD DOPE:lipid 16 (45:30:25, m/m/m) modified with different molar % of PEG²⁰⁰⁰-dialdehyde after incubation in serum.

FIG. 24b shows turbidity measurement of LD DOPE:lipid 14 (45:30:25, m/m/m) modified with different molar % of PEG²⁰⁰⁰ dialdehyde after incubation in serum.

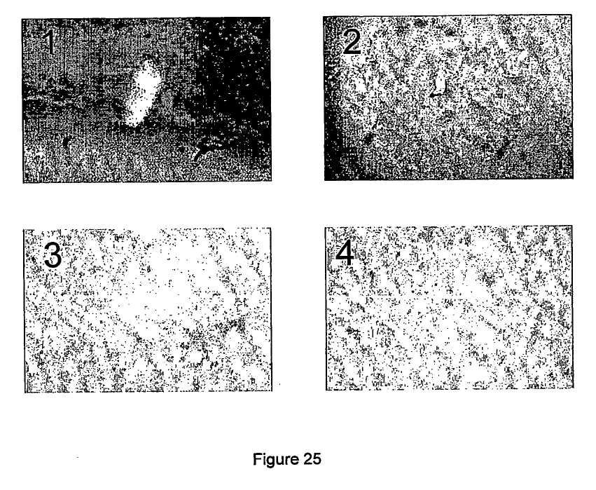

FIG. 25 pictures 1 and 3 show a microglial cell on the surface of a slice after transfection with formulation II consisting of the liposome formulation LIPIDB198/DOPE/aminoxylipid 1 (30:60:10, m/m/m). It appears that the lipoplex is trapped by phagocytosis. Picture 2 shows pyramidal neurons from the CA1 zone of the hippocampus after transfection with the formulation II. Picture 4 shows a layer of pyramidal neurons (low magnification) after transfection with formulation III.

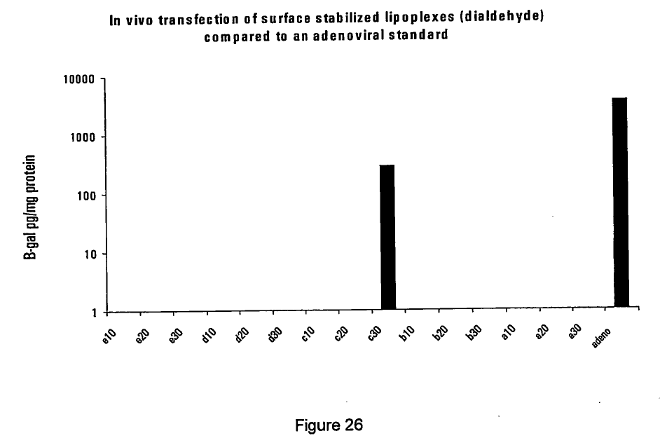

FIG. 26 shows in vivo efficacy of samples LMDa-e at 10, 20 and 30 μg/animal pDNA intranasal administration. Plasmid NGVL-1 (7.5 kb β-gal). A, μ/B198/DOPE; B μ/B198/DOPE/aminoxy lipid 1; C, μB198/DOPE/aminoxy lipid 1+5% PEG²⁰⁰⁰-dialdehyde; D, C18-μB198/DOPE/aminoxy lipid 1; E, C18-μB198/DOPE/aminoxy lipid 1+5% PEG²⁰⁰⁰-dialdehyde.

The present invention will now be described in further detail in the following examples.

### EXAMPLES

Synthetic Procedures

General. ¹H NMR spectra were recorded on either Brucker AM 500, Brucker DRX₄₀₀, Brucker DRX₃₀₀ or Jeol GX-270Q spectrometers, using residual isotopic solvent (CDCl₃, δ_{H}=7.26 ppm) as an internal reference. Mass spectra were recorded on a Micromass AutoSpecQ mass spectrometer (Brucker), or on MALDI (Brucker). HPLC (analytical and semipreparative) were run on a Hitachi (Merck) system.

Abbreviations. DIEA, diisopropylethylamine; DMF, dimethylformamide; DCM, dichloromethane; EDC, 1-ethyl-3-(3′-dimethylaminopropyl)carbodiimide hydrochloride; EDT, 1,2-ethandithiol; HBTU, O-Benzotriazole-N,N,N′,N′tetramethyl-uronium-hexafluoro-phosphate; HATU, O-7-azabentotriazol-1-yl)-N,N,N′,N′-tetramethyluronium hexafluorophosphate; MTBE, methyl-t-butylether; OpF, pentafluorphenol; PCS, photon correlation spectroscopy; TFA, trifluoroactic acid;

Targeting Compounds

General. Targeting ligands were prepared containing a folate unit covalently coupled to the y-carboxy group of folate to a free amine of an amino acid on solid phase, resulting in an amide bond between the peptide and the folic acid. Folate receptors are overexpressed on almost all cancer cell lines. A twofold strategy was applied (a) Post-coupling of the folate ligand via the thiol group of the C-terminal cysteine residue onto the malemeido group of a polyethyleneglycol unit such as OpF-acon-PEG-mal or CHO-PEG-mal and (b),(c) post-coupling via an aminoxy unit onto the second free aldehyde group of the dialdehyde, post-coupled onto the lipoplex, according to illustration in FIGS. 1B and 1C.

### Folate-(Gly)₃-(Arg)₃-(Gly)₃-Cys-OH

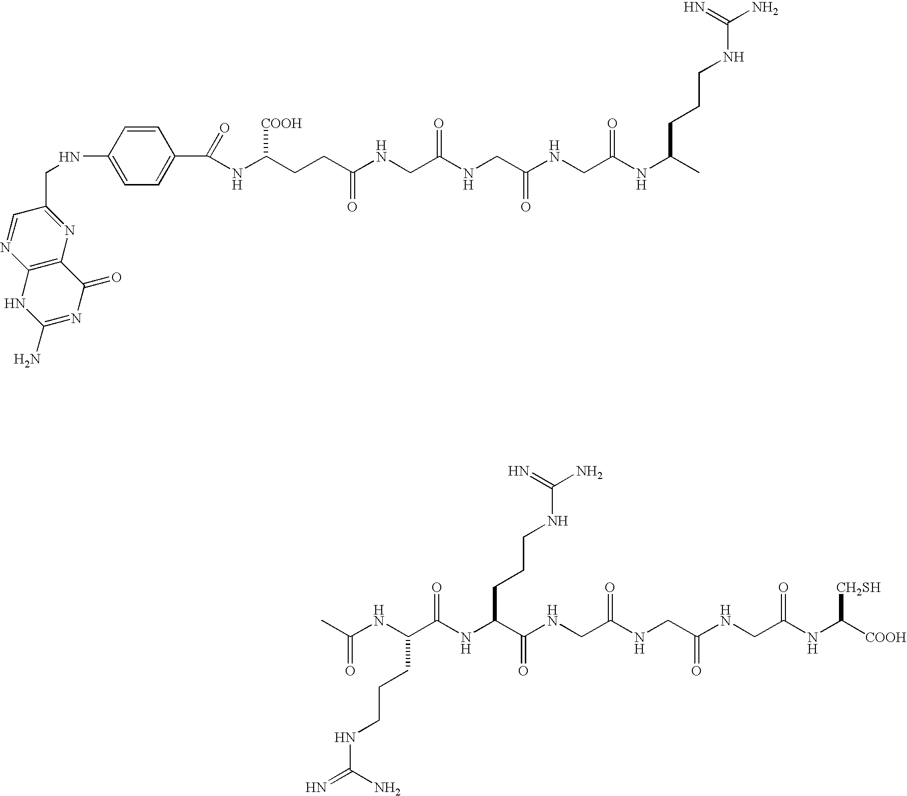

Fmoc-Cys(Trt)-Wang resin (0.53 mmol/g loading, 200 mg) were swollen in DMF for 16 h, extensively washed with DMF. Fmoc deblocking was achieved by using piperidine (20%) in DMF (2×5 mins) followed by extensive washing with DMF. For each coupling step, 3 equivalent of amino acid, 5 equivalents of DIEA and 3 equivalents of HBTU were used. Each coupling was carried out for 30 mins followed by capping with acetic anhydride (10%) in DMF in the presence of 3 equivalents of DIEA. The peptide was cleaved using 3 mL of a solution consisting of TFA (10 mL), water (0.5 mL), EDT (0.25 mL), thioanisole (0.5 mL) and phenol (0.75 g) during 3 hours. Precipitation of peptide was achieved by addition of MTBE (20 mL), followed by centrifugation at 300 rpm (20 mins). The supernatant was removed, and the yellow peptide dissolved in water (3 mL). HPLC analysis (Hitachi, C₁₈ column, gradient 0-40% acetonitrile, 40 mins, flow rate 1 mL/min) gave two major peaks absorbing at λₘₐₓ=289 nm. MALDI analysis of crude peptide gave one major ion at m/z=1355, which corresponds to [M⁺]. Purification of crude peptide was achieved on a Gilson semipreparative HPLC system using a LaChrom semipreparative C₁₈ column, flow rate 7 mL/min, monitoring wavelength A=214 nm. The existence of a free thiol was confirmed by a positive thiol test (Ellmann test, also called DTNB test: 40 mg of 5,5′-dithiobis(2-nitrobenzoic) acid dissolved in 0.1M NaH₂PO₃ buffer, pH 8, were prepared. To 1 mL of this solution, 20 μL of peptide (10 mg/mL) were added, which immediately generated a yellow colour due to the free thiol). MALDI m/z=1355.84[M⁺]. HPLC analytical (C₁₈, 0-40% acetonitrile, 40 mins, flow rate 1 mL/min, tᵣ=15 min, single peak.

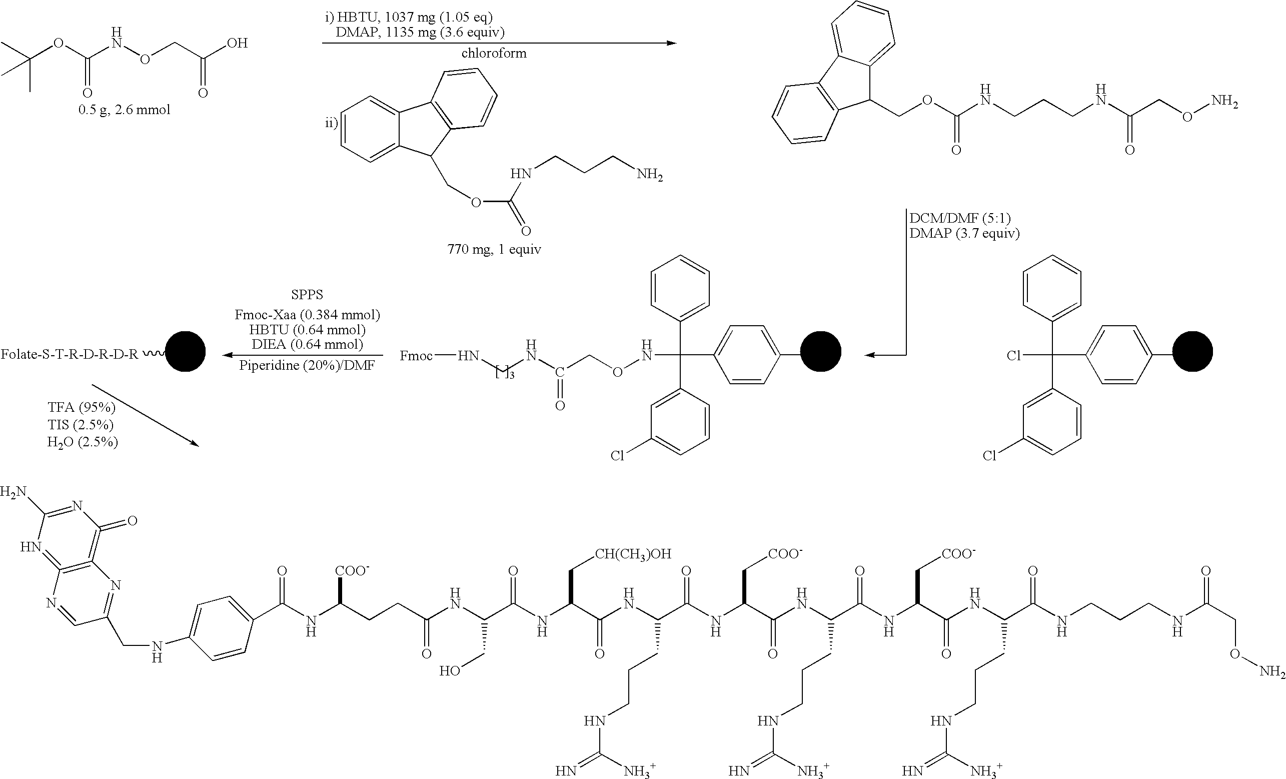

General. This targeting ligand was synthesized to couple onto the second free aldehyde function of dialdehyd-PEG²⁰⁰⁰ on the surface of LD or LMD systems as part of the post-coupling strategy depicted in FIG. 1.

Experimental Procedure A suspension of Boc-NHCO—CH₂—COOH (0.5 g, 2.6 mmol), HBTU (1037 mg, 1.05 equiv), DMAP (960 mg, 3 equiv) and Fmoc-NH—(CH₂)₃—NH₃.Cl (770 mg, 1 equiv) in DCM (50 mL) were stirred for 16 h to give a clear solution. Extraction of basic compounds was achieved using citric acid (7%, 3×100 mL), the organic phase dried over magnesium sulfate and all solvent evaporated. ¹H NMR σ(ppm) 8.4 (s, 1H, NH—CO), 8.1 (s, 1H, NH-Fmoc) 7.8 (d, 2H, 7.4 Hz, Fmoc), 7.6 (d, 2H, 7.4 Hz, Fmoc), 7.4 (d×d, 2H, 7.2 Hz, 7.2 Hz), 7.32 (d×d×d, 2H, 4 Hz, 7.4 Hz, 1.1 Hz), 5.6 (m, 1H, NH-aminoxy), 4.25 (d, 1H, Fmoc), 4.2 (s, 2H, CH₂-aminoxy), 4.1 (m, 1H, Fmoc), 1.55 (m, 6H, CH₂CH₂CH₂), 1.3 (s, 9H, Boc). Deblocking of the Boc group was achieved in TFA/water (80%) for 1.5 h. The identity of the product was confirmed by mass spectrometry (ESI), m/z=410 [M+K]⁺. HPLC (C₁₈), 0-100% CH₃CN, 40 min), t_{R}=27.52 min.

SPPS. Chlorotrytylresin (0.5 g, 1.4 mmol/g) was swollen in DCM before addition of Fmoc-NH(CH₂)₃—NHCO—CH₂—ONH₂ (320 mg, 1.15 mmol) and DMAP (320 mg, 3.7 equiv), and shaken for 3 h. Exact loading of resin was determined by UV(300 nm) using an extinction coefficient ε(Fmoc)=7800 M⁻¹cm⁻¹. A loading of 0.128 mmol was determined. For subsequent couplings of the Fmoc-amino acid, 3 equivalents of amino acid, 5 equivalent of HBTU and 5 equivalent of DIEA in DMF (10 mL) were taken, coupling time 1 h. Fmoc deblocking was achieved using piperidine in DMF (20%). Folic acid (283 mg) was dissolved in DMF (30 mL), before addition of EDC (125 mg, 1 equiv) and NHS (73 mg, 1 equiv) and DIEA (220 L, 10 equiv) and coupled for 2 h. Cleavage from the resin was achieved using TFA (4.9 mL 0.125 mL water/0.125 mL triisopropylsilane). The black solution was precipitated in MTBE to give a yellow, water-soluble precipitate, MALDI m/z 1457.89[M⁺]. This crude product was freeze-dried and purified on a Hitachi semi preparative HPLC (C₁₈), 0-100% CH₃CN, 40 min), t_{R}=18.6 min.

Derivatives of Polyethyleneglycol

General. Novel polyethyleneglycol derivatives were synthesised with a two fold aim: (i) to introduce a chemoselective moiety which selectively reacts onto the aminoxy group of aminoxylipids 1 and 26 (d) for post-coupling of these PEG derivative onto the surface of the lipoplex, and (ii) polyethyleneglycol derivatives containing an acid labile linker (cis-aconitic acid) which can be cleaved at acid pH (triggerability) (e) and (f). This second strategy shall afford an overall increased triggerable (acid labile) bond between the lipid and the PEG moiety ¹² 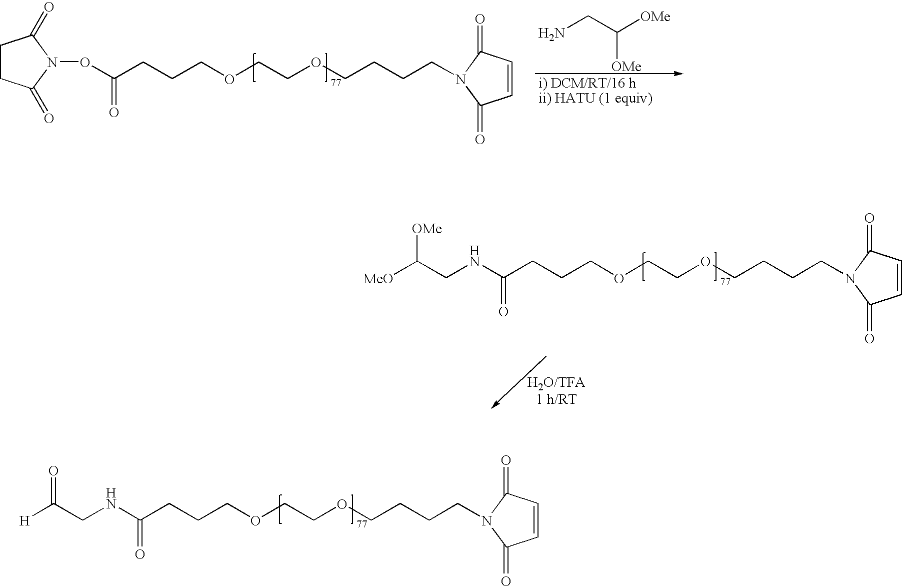
NHS-PEG-mal (100 mg, 0.029 mmol) and 1-amino-2-dimethoxy-ethane (3 equiv, 9.1 mg, 10 μL) in DCM were stirred for 1 h before addition of HATU (1 equiv, 11 mg) and stirring for 16 h, All DCM was evaporated, water (2 mL) was added and lyophilized to give a white powder, which was dissolved in water (0.6 mL). TFA (2.4 mL) was added and stirred for 1 h. The reaction mixture was frozen in liquid nitrogen and lyophilized. The oily residual was taken up in CDCl₃ which resulted in an emulsion that was lyophilized. Addition of CDCl₃ (2 mL) resulted in a clear solution. 0.8 mL of that solution was used for ¹H NMR: a huge resonance at 9 ppm (aldehyde) indicated that there was still excess starting material present. Slow addition of MTBE to the combined solutions of chloroform resulted in a white precipitate, which was centrifuged (3000 rpm/5 mins), the supernatant removed, the residual taken up in water, lyophilized. 22 mg of white powder were dissolved in CDCl₃ (0.8 mL) for an ¹H NMR analysis: σ(ppm) 12 (0.5H, COOH), 9.6 (s, 1H, CHO), 9(6.1H, CHO starting material), 7.3 (CHCl₃), 6.7 (s, 1H, mal), 6.6 (s, 0.1H, mal), 6.5 (s, 1H, mal), 3.5 (s, 150H, CH₂CH₂O-PEG), 2.9 (s, broad, 4-6H, CH2CH2-mal), 1.1 (s, 3-5H, —CO—CH₂—CH₂CH₂—O—). The ¹H NMR indicated that the right product was obtained with an estimated purity of ≈80%, whereas the rest is the free carboxylic acid cpd. In order to remove remaining traces of TFA and starting material, the product was lyophilized another two times, which resulted in a nylon-like white polymer, insoluble in water. It is likely that the aldehyde under neutral/basic conditions polymerized.

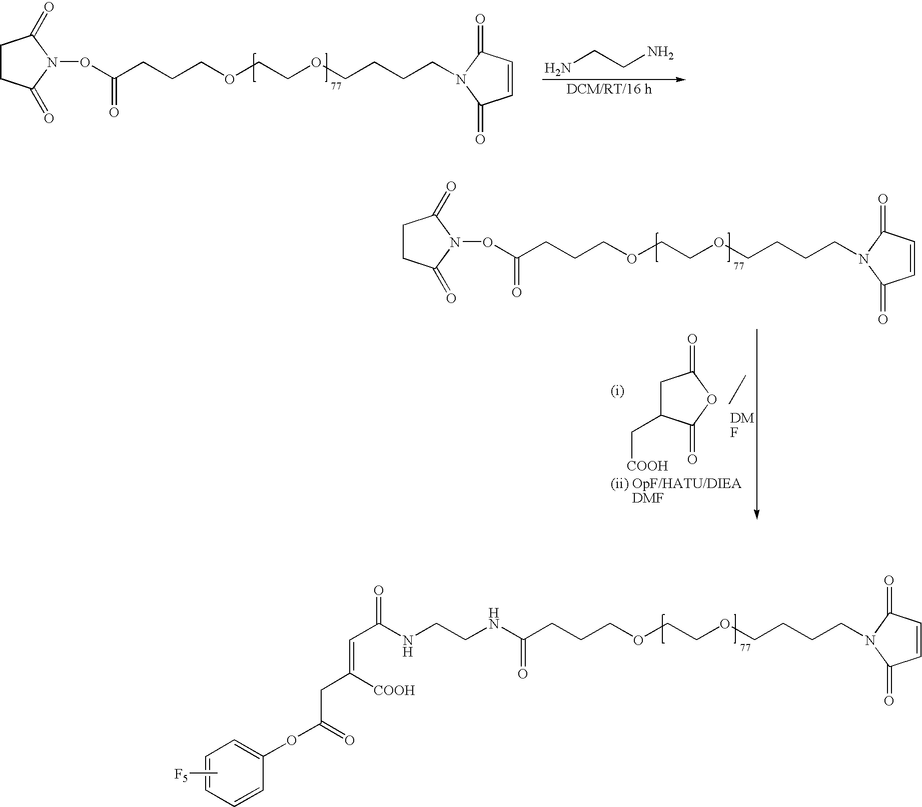

To a solution of 100 mg NHS-PEG³⁴⁰⁰-mal (Shearwaters, USA) in 390 mL DCM, 1,2-diamino-ethane (390 μL) in DCM (10 mL) were added dropwise over 30 mins and stirred for 1 h/RT. 20 mg HBTU (Advanced Chem Tech, UK) were added to drive the reaction to completion, and stirred for 16 h/RT. All DCM was evaporated, and the residual taken up in water (2 mL) and acetonitrile (5 mL), the clear solution frozen in liquid nitrogen and freeze dried, to give a white powder, which easily dissolved in water. The product was lyophilized a second time before dissolving in DMF (2 mL). DIEA (25 μL) and cis-aconitate anhydride (6 equiv, 27 mg) (Sigma, UK) were added and the reaction stirred for 16 h. Work-up: Water (20 mL) was added and extracted with diethylether (3×50 mL). The water layer was separated from the organic layers and lyophilized. A sample of this product was analysed by analytical HPLC (C₄, 0-100 CH₃CN, no TFA): Three peaks at t_{R}=26 min, 30 mins (major) and 33 mins. The peak at 30 mins was isolated and analysed by MALDI: m/z=3922[M⁺]; 3922±44×n (±10>n=heterogeneity of polyethyleneglycol). 75 mg (≈0.0145 mmol) of cis-acon-PEG³⁴⁰⁰-mal were dissolved in DMF (4 mL) before addition of pentafluorophenol (5 equiv, 14 mg), HATU (PE Biosystems, UK; 5 equiv, 28 mg) and DIEA (15 equiv, 38 μL) and stirred or 16 h/RT. Work-up: water (20 mL) was added and extracted with diethylether (2×25 mL), ethylacetate/diethylether (1:1; 1×50 mL) and the water phase lyophilized to give a red/brown powder. Dichloromethane (2 mL) was added to dissolve the compound, before dropwise addition of MTBE to give a red/brown precipitate. The precipitate was collected by centrifugation (3000 rpm/5 min), taken up in water and lyophilized to give a red/brown powder. 22 mg were dissolved in CDCl₃ and analyzed by ¹H-NMR. σ(ppm) 8.5 (s, 2H, CONH), 7.4 (s, CHCl₃), 6.5 (s, 1H, acon-CH═C), 6.4 (s, broad, mal-H), 5.8 (s, broad, mal-H′), 4 (s, 150H, CH₂CH₂O-PEG), 3.5 (s, 2H, CH₂CH₂—NH), 3.2 (2H, s, 2H, CH₂CH₂—NH), 1.3 (s, broad, 4H, CH₂CH₂-mal). No mass could be detected by ESI and MALDI due to the blockage of all ionisable groups. HPLC analytical (C₄, 0-100 CH₃CN, no TFA), t_{R}=21-34 min (broad peak typical for PEG).

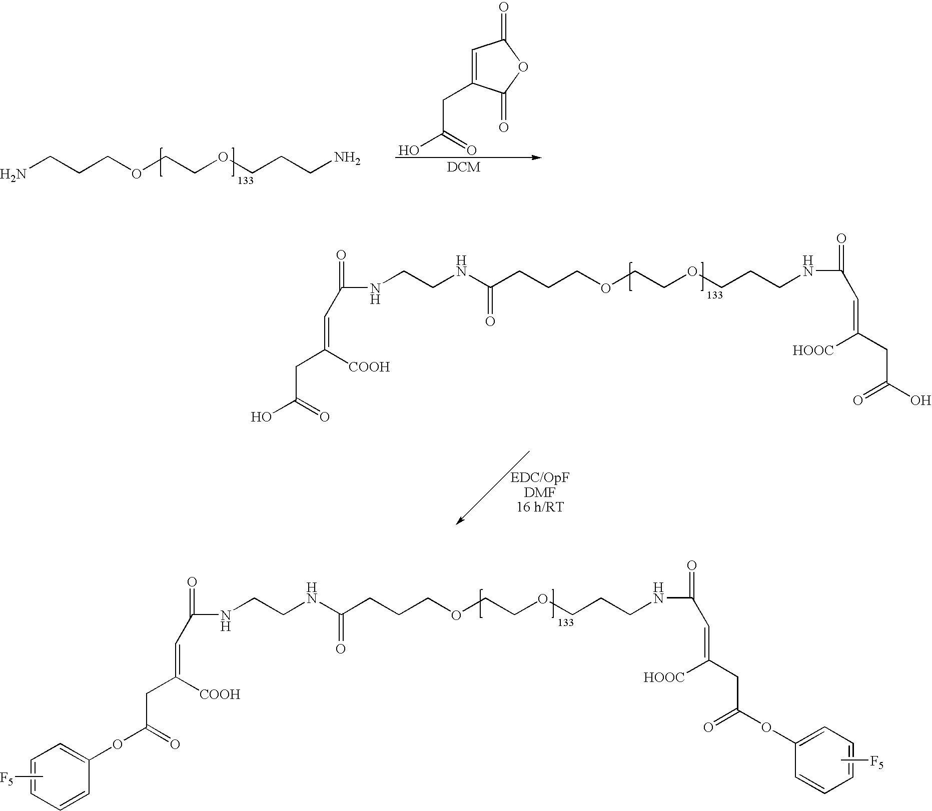

NH₂—PEG6000-NH₂ (100 mg, 0.0167 mmol) were dissolved in DCM (2 mL) before addition of cis-aconitic anhydride (10 equiv, 26 mg) and stirred for 2 h to give a yellow solution which turned into red after 4 h. All solvent was evaporated, water added 10 mL and excess cis-aconitic anhydride extracted with diethylether (3×20 mL), and the water lyophilized to give a yellow/red powder. HPLC indicated that the reaction did not go to completion. The powder was dissolved in DMF (2 mL), DIEA (35 μL) and cis-aconitic anhydride (26 mg) added and the reaction mixture heated at 50° C./3 h, and worked-up as described. To the red/brown powder dissolved in DMF (2 mL) was added EDC (12.7 mg, 4 equiv) and OpF (12.2 mg, 4 equiv) and stirred for 16 h/RT. Work-up: Water (10 mL) was added, and extracted with diethylether (3×20 mL). The water was frozen and lyophilized to give a white powder, which was dissolved in DCM (2 mL). Addition of MTBE resulted in a red/brown precipitate, which was centrifuged (3000 rpm, 5 mins), the supernatant removed and the residual taken up in water (2 mL), lyophilized. The red/brown powder was dissolved in DCM (2 mL). Addition of MTBE resulted in a red/brown precipitate, which was centrifuged (3000 rpm, 5 mins), the supernatant removed and the residual taken up in water (2 mL), and lyophilized to give a red/brown powder.

Cholesterol Derivatives

Synthesis of Triggerable Lipids

Summary of Syntheses. A variety of cholesterol based cationic and neutral lipids were prepared, suitable for the post-coupling strategy with polyethyleneglycol derivatives. Four lipids served as general starting points for further modification: First, (2-aminoethyl)carbamic acid cholesteryl ester (21) and secondly, 4-aza-(tert-butoxycarbonyl)-N⁶(cholesteryloxycarbonylamino) hexlyamine (8). Each 01 and 8 were then further modified to serine (13, 14) and cysteine (15, 16) containing lipids, respectively. Lipid 01 was modified to neutral aminoxylipid 19, whereas lipid 8 was further modified to charged aminoxylipid 26. The third principal lipid, the glycyl-cholesteryl-lipid 20, was modified to the hydrazide lipid 23. Finally, the fourth starting lipid, cholesteryl-carbamate, was modified to the hydrazone lipid 24. for an Overview, see table 1.

TABLE 1

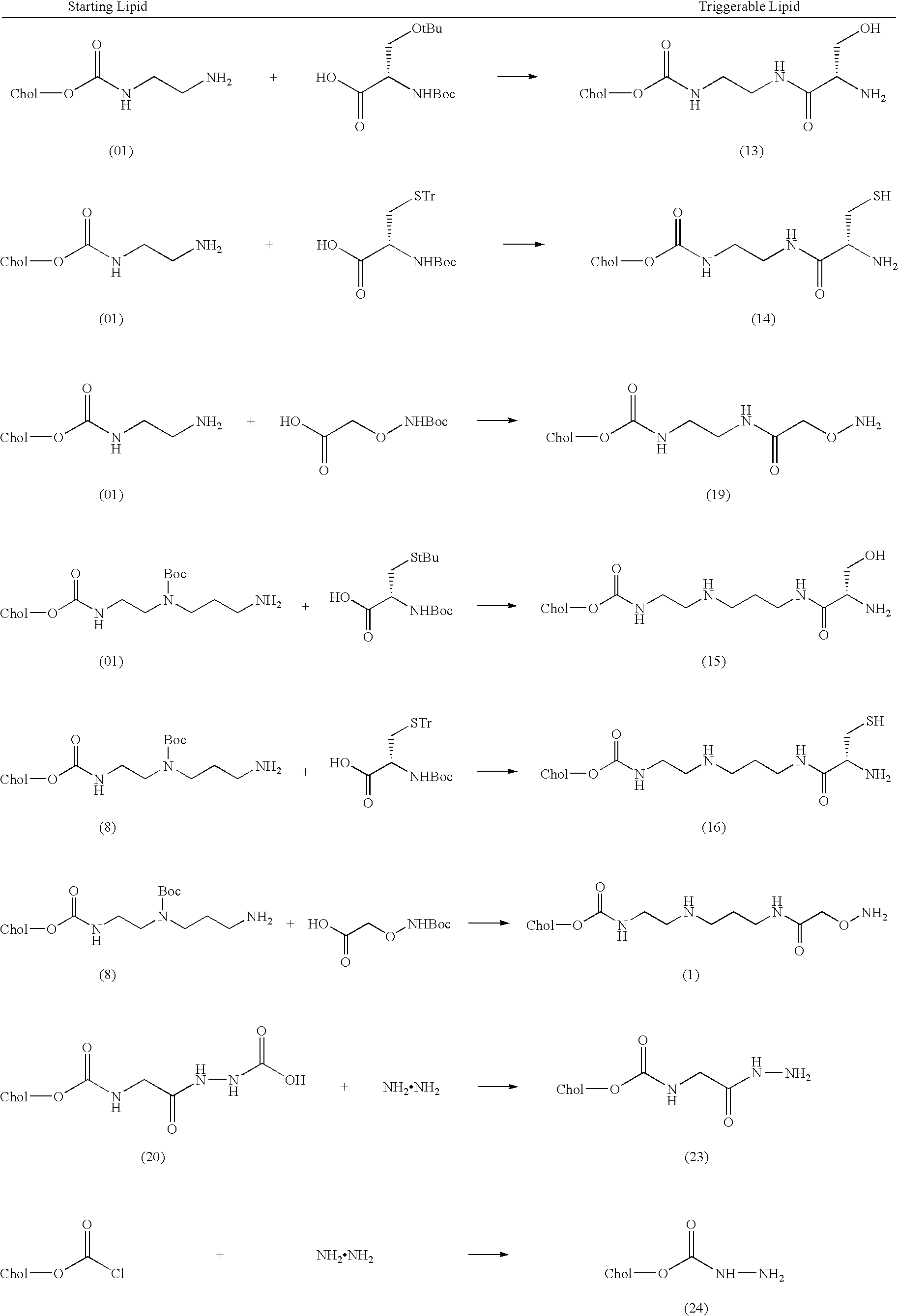

Synthetic Procedures. Dried CH₂Cl₂ was distilled with phosphorous pentoxide, other solvents were purchased pre-dried as required. Thin layer chromatography (Tlc) was performed on pre-coated Merck-Kieselgel 60 F₂₅₄ aluminium backed plated and revealed with ultraviolet light, iodine, acidic ammonium molybdate (IV), acidic ethanolic vanillin, or other agents as appropriate. Flash column chromatography was accomplished on Merck-Kieselgel 60 (230-400 mesh) with convenient solvent visualised with ultraviolet light (254 nm), iodine, acidic molybdate (IV), acidic ethanolic vanillin, aqueous potassium manganate (VII), 4,4′-bis(dimethylamino)benzylhydrol in acetone or iodine as appropriate. Infrared Spectra were recorded on Jasco FT/IR 620 using NaCl plates. Mass spectra (Positive ions electrospray) were recorded using VG-7070B or JEOL SX-102 instruments. ¹H & ¹³C NMR spectra were recorded on either Bruker DRX300, DRX400 or Jeol GX-270Q machines using residual isotopic solvent as an internal reference.

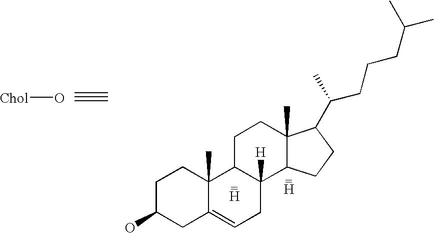

### (2-Aminoethyl)carbamic Acid Cholesteryl Ester (01)

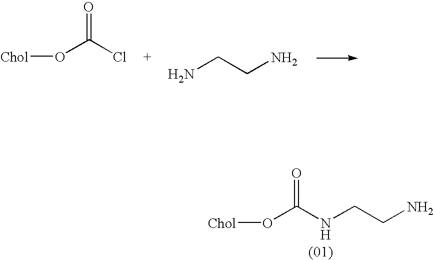

Cholesteryl chloroformate (7.5 g, 0.0167 mol) was dissolved in ethylene-1,2-diamine (180 ml) and the mixture stirred for 15 h. The reaction was quenched with water and extracted with dichloromethane. The organic extracts were dried (MgSO₄) and the solvent removed in vacuo to afford a residue which was purified by flash column chromatography giving the pure title compound 01 (5.5 g, 0.0116, 73%).

### 2-(Cholesteryloxycarbonyl)aminoethanol (2)

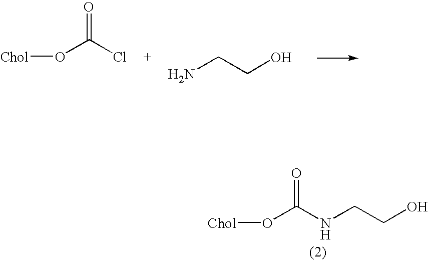

Ethanolamine (15 ml, 0.246 mol, 2.2 eq) was dissolved in DCM (35 ml) and was cooled to 0° C. using an ice bath. A solution of cholesteryl chloroformate (50 g, 0.112 mol, 1 eq) in DCM (300 ml) was added dropwise over an hour during that time a white precipitate formed. The reaction was allowed to warm to room temperature and continued stirring for 18 hours. The precipitate was removed by filtration and the solution was washed with saturated NaHCO₃ (2×75 ml), water (2×75 ml), dried (MgSO₄) and the solvent removed under reduced pressure to give 2-(Cholesteryloxycarbonyl)aminoethanol 2 (44 g, 87%). δ_{H} (300 MHz) 5.39 (1H, m, H-6), 5.02 (1H, m, N—H), 4.52 (1H, m, H-3), 3.74 (2H, t, J=5.5 Hz, H-2′), 3.35 (2H, t, J=5 Hz, H-1′), 2.38-2.25 (2H, m, H-4), 2.08-1.72 (5H, m, H-2, H-7, H-8), 1.64-1.05 (21H, m, H-1, H-9, H-11, H-12, H-14, H-15, H-16, H-17, H-20, H-22, H-23, H-24, H-25), 1.02 (3H, s, H-19), 0.93 (3H d, J=6.5 Hz, H-21), 0.89 (6H, dd, J 1 Hz 6.5 Hz, H-26, H-27), 0.69 (3H, s, H-18). m/z (FAB⁺) 469 (M+Na)⁺, 474 (M+H)⁺, 369 (Chol)⁺.

### 2-[(cholesteryloxycarbonyl)amino]ethyl Methanesulfonate (3)

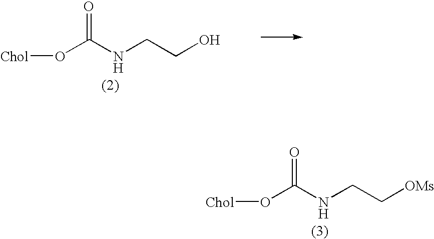

To a solution of 2-[(cholesteryloxycarbonyl)amino]ethanol 2 (0.45 g, 0.96 mmol, 1.0 eq) and triethylamine (0.4 ml, 2.88 mmol, 3.0 eq) in DCM (10 ml) at 0° C. was added dropwise a solution of methanesulfonyl chloride (0.19 ml, 2.40 mmol, 2.5 eq). The reaction was allowed to warm to room temperature and stirred for 30 minutes. After tlc indicated the reaction was complete, ice was added to quench the reaction the reaction mixture was then poured into saturated aqueous NH₄Cl (15 ml) and extracted with ether (3×10 ml), brine (1×10 ml) and dried (Na₂SO₄). The solvent was removed under reduced pressure to give a white solid which on purification by chromatography (ether) gave 2-[(cholesteryloxycarbonyl)amino]ethyl methanesulfonate 3 (0.48 g, 90%). δ_{H} (300 MHz) 5.39 (1H, d, J=5 Hz, H-6), 5.00 (1H, m, N—H), 4.52 (1H, m, H-3), 4.32 (2H, t, J=5 Hz, H-2′), 3.55 (2H, m, H-1′), 3.06 (3H, s, OMs), 2.36-2.29 (2H, m, H4), 2.04-1.81 (5H, m, H-2, H-7, H-8), 1.64-1.05 (21H1, m, H-1, H-9, H-11, H-12, H-14, H-15, H-16, H-17, H-20, H-22, H-23, H-24, H-25), 1.02 (3H, s, H-19), 0.93 (3H d, J=6.5 Hz, H-21), 0.89-0.87 (6H, dd, J 1 Hz 6.5 Hz, H-26, H-27), 0.69 (3H, s, H-18). m/z (FAB⁺) 574 (M+Na)⁺, 552 (M+H)⁺, 369 (Chol)⁺.

### 4-aza-N⁶(cholesteryloxycarbonylamino)hexanol (4)

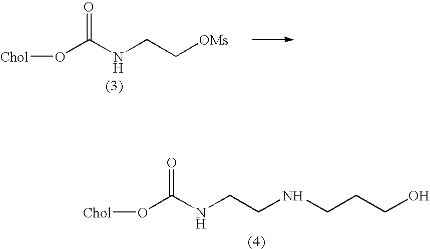

A round bottomed flask was charged with 2-[(cholesteryloxycarbonyl)amino]ethyl methanesulfonate 3 (15.6 g, 0.029 mol, 1.0 eq) and 3 amino-butan-1-ol (150 ml, 7.5 mmol, 10 eq). Once tlc indicated the reaction was complete (approximately 3 days) DCM (100 ml) and K₂CO₃ (6 g) were added and stirred for 30 minutes. The suspension was then passed through a short pad of Celite® washing thoroughly with DCM, ethanol and 10% NEt₃/EtOH. The solvent was removed under reduced pressure to give a yellow oil. This was redissolved in DCM (10 ml) and washed with water (3×3 ml), brine (3 ml) and dried (Na₂SO₄). The solvent was removed in vacuo and purified by chromatography to give 4-aza-N⁶ (cholesteryloxycarbonylamino)hexanol 4 (12.45 g, 81%). δ_{H} (300 z and 270 MHz) 5.38 (1H, m, H-6), 4.48 (1H, m, H-3), 3.77 (2H, t, J=5 Hz, H-5′), 3.26 (2H, m, H-1′), 2.91 (2H, t, J=6 Hz, H-2′), 2.82 (2H, t, J=6 Hz, H-3′), 2.30-2.23 (2H, m, H-4), 2.00-1.76 (5H, m, H-2, H-7, H-8), 1.74-1.00 (23H, m, H-4′, H-1, H-9, H-11, H-12, H-14, H-15, H-16, H-17, H-20, H-22, H-23, H-24, H-25), 0.99 (3H, s, H-19), 0.92-0.90 (3H d, J=6 Hz, H-21), 0.87-0.85 (6H, dd, J 1Hz 6 Hz, H-26, H-27), 0.68 (3H, s, H-18). m/z (FAB⁺) 543 (M+Na)⁺, 531 (M+H)⁺, 369 (Chol)+145, 105, 91 (C₇H₇)⁺, 81 (C₆H₉)⁺, 55.

### 4-aza-(tert-butoxycarbonyl)-N⁶(cholesteryloxycarbonylamino)hexanol (5)

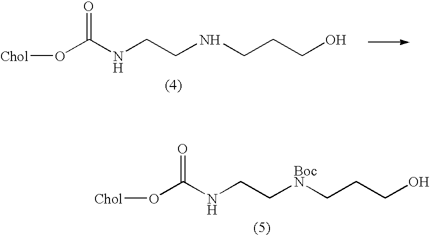

To a solution of 4-aza-N⁶(cholesteryloxycarbonylamino)hexanol 4 (3 g, 5.64 mmol, 1 eq) and di-tert-butyl-dicarbonate (1.26 g, 5.64 mmol, 1.0 eq) in DCM (18 ml), was added NEt₃ (0.9 ml, 6.18 mmol, 1.1 eq) and the resulting solution observed by tlc. On completion the reaction mixture was poured into saturated aqueous NH₄Cl (15 ml) and extracted with DCM (2×40 ml). The combined organic extracts were washed with water (3×40 ml) and dried (Na₂SO₄). The solvent was removed in vacuo to give 4-aza-(tert-butoxycarbonyl)-N⁶(cholesteryloxycarbonylamino) hexanol 5 (3.19 g, 90%). δ_{H} (270 MHz) 5.36 (1H, m, H-6), 4.48 (1H, m, H-3), 3.53 (2H, t, J=5 Hz, H-5′), 3.40-3.25 (6H, m, H-1′, H-2′, H-3′), 2.30 (2H, m, H-4), 2.00-1.70 (5H, m, H-2, H-7, H-8), 1.05 (9H, s, Boc Hs 3×CH_{3),} 1.60-1.00 (23H, m, H-4′, H-1, H-9, H-11, H-12, H-14, H-15, H-16, H-17, H-20, H-22, H-23, H-24, H-25), 0.98 (3H, s, H-19), 0.93-0.90 (3H d, J=6 Hz, H-21), 0.88-0.86 (6H, dd, J 1 Hz 6 Hz, H-26, H-27), 0.65 (3H, s, H-18). m/z (FAB⁺) 643 (M+Na)⁺, 631 (M+H)⁺, 531 (M-Boc), 369 (Chol)⁺,163, 145, 109, 91 (C₇H₇)⁺, 81 (C₆H₉)⁺, 57.

### 4-aza-(tert-butoxycarbonyl)-N⁶(cholesteryloxycarbonylamino)hexyl methanesulfonate (6)

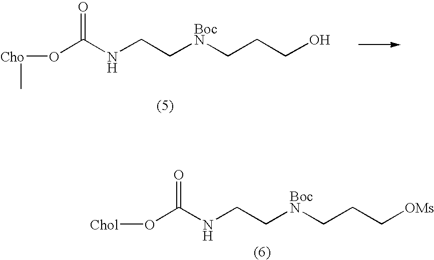

This preparation was carried out as described earlier in the preparation of 2-[(cholesteryloxycarbonyl)amino]ethyl methanesulfonate 2 on a 0.0114 mol scale giving after chromatography (ether), 4-aza-(tert-butoxycarbonyl)-N⁶ (cholesteryloxycarbonylamino) hexyl methanesulfonate 6 (0.73 g, 0.87%). δ_{H} (300 z) 5.38 (1H, m, H-6), 4.49 (1H, m, H-3), 4.41 (2H, t, J=6 Hz, H-5′), 4.29 (2H, t, J=5 Hz, H-2′), 3.55 (2H, m, H-1′), 3.55-3.35 (2H, m, H-3′), 3.16 (3H, s, OMs CH₃), 2.35 (2H, m, H-4), 2.12-1.70 (5H, m, H-2, H-7, H-8), 1.38 (9H, s, Boc Hs 3×CH₃), 1.67-1.00 (23H, m, H-4′, H-1, H-9, H-11, H-12, H-14, H-15, H-16, H-17, H-20, H-22, H-23, H-24, H-25), 0.96 (3H, s, H-19), 0.93-0.91 (3H d, J=6 Hz, H-21), 0.88-0.86 (6H, dd, J 1 Hz 6 Hz, H-26, H-27), 0.69 (3H, s, H-18). m/z (FAB⁺) 609 (M−Boc), 369 (Chol)⁺, 145, 121, 105, 95 (C₇H₁₁)⁺, 81 (C₆H₉)⁺, 69, 55.

### 4-aza-(tert-butoxycarbonyl)-N⁶(cholesteryloxycarbonylamino)hexazide (7)

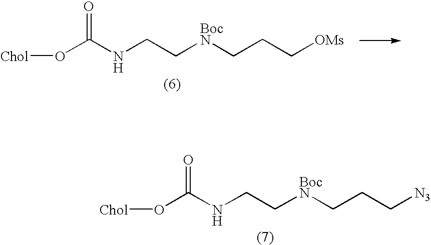

4-aza-(tert-butoxycarbonyl)-N⁶(cholesteryloxycarbonylamino)hexyl methanesulfonate (7.0 g, 9.88 mmol, 1 eq), sodium azide (3.2 g, 0.049 mol, 5 eq) and sodium iodide (1.56 g, 9.88 mmol, 1.0 eq) were all placed under nitrogen in the round bottomed flask. Anhydrous DMF (50 ml) was added with stirring and a reflux condenser fitted and heated at 80° C. for 5.5 hours. Once tlc indicated that the reaction had gone to completion the flask was allowed to cool to room temperature, the DMF removed under reduced pressure and the residue then redissolved in EtOAc. This was washed with sodium hydrogen carbonate (2×50 ml), water (2×10 ml), brine (50 ml) and dried (Na₂SO₄). The solvent was then removed under reduced pressure and purified by chromatography (petrol 1: ether 1) to give 4-aza-(tert-butoxycarbonyl)-N⁶(cholesteryloxycarbonylamino) hexanamine 7 (5.7 g, 88%). δ_{H} (300 MHz) 5.38 (1H, m, H-6), 4.85 (1H, m, N—H), 4.53-4.50 (1H, m, H-3), 3.38-3.28 (8H, m, H—S′, H-3′, H-2′, H-1′), 2.36-2.25 (2H, m, H-4), 1.90-1.78 (5H, m, H-2, H-7, H-8), 1.48 (9H, s, Boc Hs 3×CH₃), 1.63-1.05 (23H, m, H-4′, H-1, H-9, H-11, H-12, H-14, H-1S, H-16, H-17, H-20, H-22, H-23, H-24, H-25), 1.01 (3H, s, H-19), 0.93-0.91 (3H d, J=6 Hz, H-21), 0.88-0.86 (6H, dd, J 1 Hz 6 Hz, H-26, H-27), 0.68 (3H, s, H-18). m/z (FAB⁺) 656 (M+H)⁺, 556 (M−Boc), 369 (Chol)⁺, 145, 121, 105, 95 (C₇H₁₁)⁺, 81 (C₆H₉)⁺, 57.

### 4-aza-(tert-butoxycarbonyl)-N⁶(Cholesteryloxycarbonylamino)-hexlyamine (8)

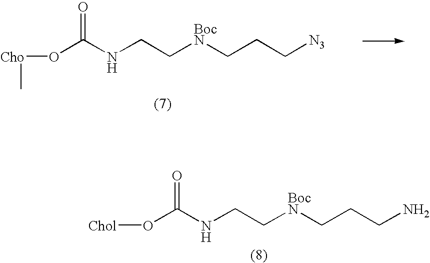

To a stirred solution of 4-aza-(tert-butoxycarbonyl)-N⁶(cholesteryloxycarbonylamino) hexanamine 7 (2.0 g, 3.05 mmol, 1 eq) dissolved in THF (22 ml), trimethylphosphine (3.51 mmol, 1.15 eq) in THF (3.5 ml) was added. Once tlc indicated that the reaction had gone to completion, water (3.5 ml) and aqueous ammonia (3.5 ml) were added and stirred for a further hour. The solvent was removed in vacuo. Purification by chromatography (ultra/2) gave 4-aza-(tert-butoxycarbonyl)-N⁶(cholesteryloxycarbonylamino) hexlyamine 8 (1.44 g, 76%) as a white solid. δ_{H} (270 MHz, CHCl₃) 5.36 (1H, m, H-6), 4.464.44 (1H, m, H-3), 3.31-3.22 (6H, m, H-3′, H-2′, H-1′), 2.67 (2H, t, J=6 Hz, H-5′), 2.29 (2H, m, H-4), 2.05-1.79 (5H, m, H-2, H-7, H-8), 1.45 (9H, s, Boc Hs 3×CH₃), 1.78-1.05 (23H, m, H-4′, H-1, H-9, H-11, H-12, H-14, H-15, H-16, H-17, H-20, H-22, H-23, H-24, H-25), 0.98 (3H, s, H-19), 0.91-0.88 (3H d, J=6 Hz, H-21), 0.86-0.83 (6H, dd, J 1 Hz 6 Hz, H-26, H-27), 0.66 (3H, s, H-18). m/z (FAB⁺) 630 (M+H)⁺, 530 (M−Boc), 369 (Chol)⁺, 145, 121, 109, 95 (C₇H₉)⁺, 81 (C₆H₉)⁺,61, 57.

Protected Serine Derivative (9)

N-α-Boc-O-tert-butyl-L-serine (74 mg, 0.281 mmol) in anhydrous dichloromethane was treated successively with DMAP (40 mg, 0.324 mmol), HBTU (128 mg, 0.337 mmol) and amine 01 (100 mg, 0.216 mmol) and the mixture stirred at r.t. under a nitrogen atmosphere for 15 h. The reaction was quenched with water and extracted with dichloromethane. The dried (MgSO₄) extracts were concentrated in vacuo to afford a residue which was purified by flash column chromatography affording pure 9 (0.149 mmol, 69%). δ_{H} (270 MHz, CHCl₃) 6.7 (1H, br s), 5.3 (2H, m), 5.0 (1H, br s), 4.4 (1H, m), 4.1 (1H, m), 3.7 (1H, m), 3.2-3.4(5 h, m), 2.29 (2H, m), 2.05-1.79 (5H, m), 1.45 (9H, s, Boc), 1.15 (9H, s), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 717 (M+H)⁺, 369 (Chol).
Protected Cysteine Derivative (10)

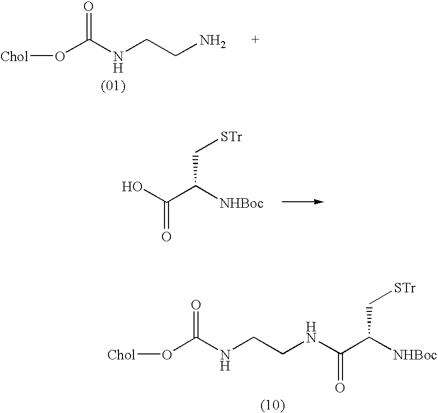

N-α-Boc-S-trityl-L-cysteine (319 mg, 0.689 mmol) in anhydrous dichloromethane was treated successively with DMAP (195 mg, 1.6 mmol), HBTU (311 mg, 0.82 mmol) and amine 01 (250 mg, 0.53 mmol) and the mixture stirred at r.t. under a nitrogen atmosphere for 15 h. The reaction was quenched with water and extracted with dichloromethane. The dried (MgSO₄) extracts were concentrated in vacuo to afford a residue which was purified by flash column chromatography affording pure 10 (0.517 mmol, 98%). δ_{H} (270 MHz, CHCl₃) 7.2-7,5 (15H, m), 6.3 (1H, br s), 5.3 (1H, m), 5.0 (1H, br s), 4.8 (1H, br s), 4.4 (1H, m), 3.7 (1H, m), 3.2-3.4 (4H, m), 2.7 (1H, m), 2.5 (1H, m), 2.29 (2H, m), 2.05-1.79 (5H, m), 1.45 (9H, s, Boc), 1.15 (9H, s), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 940.5 (M+Na)⁺, 369 (Chol).
Protected Serine Derivative (11)

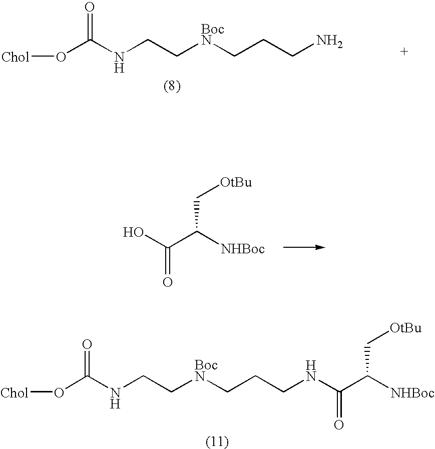

N-α-Boc-O-tert-butyl-L-serine (41 mg, 0.155 mmol) in anhydrous dichloromethane was treated successively with DMAP (66 mg, 0.54 mmol), HBTU (0.180 mmol) and amine 8 (75 mg, 0.119 mmol) and the mixture stirred at r.t. under a nitrogen atmosphere for 15 h. The reaction was quenched with water and extracted with dichloromethane. The dried (MgSO₄) extracts were concentrated in vacuo to afford a residue which was purified by flash column chromatography affording pure 11 (0.090 mmol, 76%). δ_{H} (270 MHz, CHCl₃) 6.5 (1H, br s), 5.2-5.5 (2H, m), 5.15 (1H, br s), 4.8 (1H, m), 4.4 (1H, m), 4.1 (1H, m), 3.7 (1H, m), 3.2-3.4 (9H, m), 2.29 (2H, m), 2.05-1.79 (5H, m), 1.45 (9H, s, Boc), 1.43 (9H, s, Boc), 1.15 (9H, s), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 874 (M+H)⁺, 369 (Chol).
Cysteine Derivative (12)

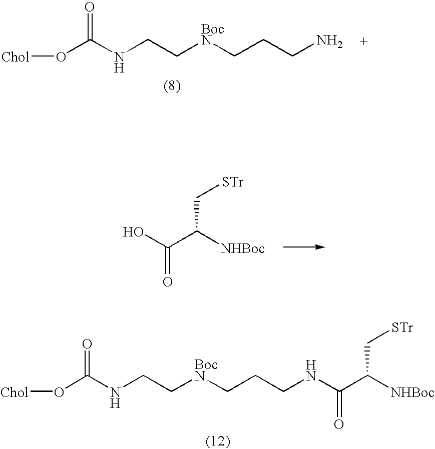

N-α-Boc-S-trityl-L-cysteine (359 mg, 0.77 mmol) in anhydrous dichloromethane was treated successively with DMAP (220 mg, 1.8 mmol), HBTU (352 mg, 0.93 mmol) and amine 8 (270 mg, 0.43 mmol) and the mixture stirred at r.t. under a nitrogen atmosphere for 15 h. The reaction was quenched with water and extracted with dichloromethane. The dried (MgSO₄) extracts were concentrated in vacuo to afford a residue which was purified by flash column chromatography affording pure 12 (0.393 mmol, 91%). δ_{H} (270 MHz, CHCl₃) 7.2-7,5 (15H, m), 6.3 (1H, br s), 5.3 (1H, m), 5.0 (1H, br s), 4.8 (1H, br s), 4.4 (1H, m), 3.7 (1H, m), 3.2-3.4 (8H, m), 2.7 (1H, m), 2.5 (1H, m), 2.29 (2H, m), 2.05-1.79 (5H, m), 1.45 (9H, s, Boc), 1.15 (9H, s), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 1097.5 (M+Na)⁺, 369 (Chol).

### Serine Derivative (13) of (2-aminoethyl)carbamic Acid Cholesteryl Ester

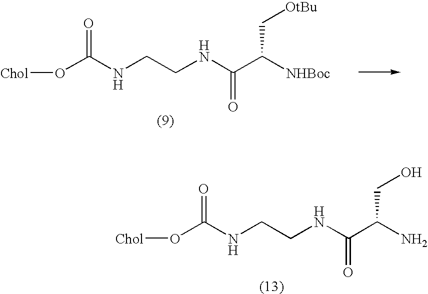

Compound 9 (100 mg, 0.14 mmol) was dissolved in a mixture of trifluoroacetic acid (18 ml), dichloromethane (5 ml) and triisopropylsilane (2 ml) and the resultant solution stirred at r.t. for 2 h. the solution was concentrated in vacuo to afford a residue which was purified by flash column chromatography affording pure 13 (0.11 mmol, 79%). δ_{H} (270 MHz, CHCl₃) 7.8 (1H, br s), 5.3 (1H, m), 5.0 (1H, br s), 4.4 (1H, m), 3.85 (1H, m), 3.65 (1H, m), 3.2-3.4 (5H, m), 2.29 (5H, m), 2.05-1.79 (7H, m), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 560.2 (M+H)⁺, 369 (Chol).

### Cysteine Derivative (14) of (2-aminoethyl)carbamic Acid Cholesteryl Ester

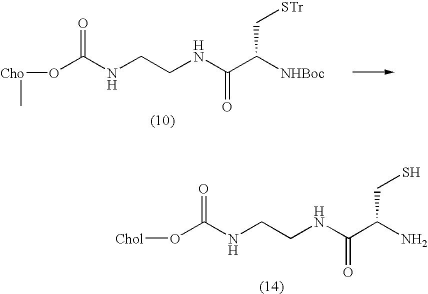

Compound 10 (420 mg, 0.457 mmol) was dissolved in a mixture of trifluoroacetic acid (18 ml), dichloromethane (5 ml) and triisopropylsilane (2 ml) and the resultant solution stirred at r.t. for 2 h. the solution was concentrated in vacuo to afford a residue which was purified by flash column chromatography affording pure 14 (0.224 mmol, 49%) δ_{H} (270 MHz, CHCl₃) 7.7 (1H, br s), 5.3 (1H, m), 5.0 (1H, br s), 4.4 (1H, m), 4.1 (1H, in), 3.6 (1H, m), 3.2-3.4 (4H, m), 2.93 (1H, m), 2.87 (1H, m), 2.29 (4H, m), 2.05-1.79 (5H, m), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 616.3 (M+K)⁺, 369 (Chol).

### Serine Derivative (15) of (2-aminoethyl)carbamic Acid Cholesteryl Ester

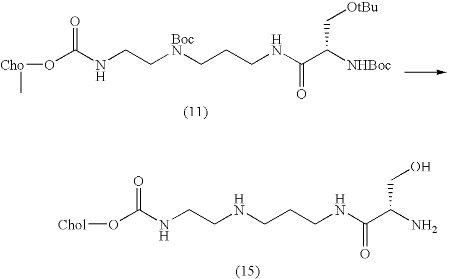

Compound 11 (70 mg, 0.08 mmol) was dissolved in a mixture of trifluoroacetic acid (18 ml), dichloromethane (5 ml) and triisopropylsilane (2 ml) and the resultant solution stirred at r.t. for 2 h. the solution was concentrated in vacuo to afford a residue which was purified by flash column chromatography affording pure 15 (0.046 mmol, 58%). δ_{H} (270 MHz, CHCl₃) 8.3 (1H, br s), 5.3 (1H, m), 5.0 (1H, br s), 4.4 (1H, m), 3.9 (1H, m), 3.8 (1H, m), 3.6 (1H, m), 3.2-3.4 (8H, m), 2.29 (5H, m), 2.05-1.79 (7H, m), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 617 (M+H)⁺, 369 (Chol).

### Cysteine Derivative (16) of (2-aminoethyl)carbamic Acid Cholesteryl Ester

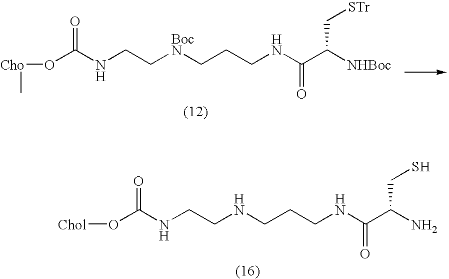

Compound 12 (390 mg, 0.363 mmol) was dissolved in a mixture of trifluoroacetic acid (18 ml), dichloromethane (5 ml) and triisopropylsilane (2 ml) and the resultant solution stirred at r.t. for 2 h. the solution was concentrated in vacuo to afford a residue which was purified by flash column chromatography affording pure 16 (0.243 mmol, 67%). δ_{H} (270 MHz, CHCl₃) 8.0 (1H, br s), 5.3 (1H, m), 5.0 (1H, br s), 4.4 (1H, m), 3.6 (1H, m), 3.2-3.4 (8H, m), 2.96 (1H, m), 2.89 (1H, m), 2.29 (5H, m), 2.05-1.79 (7H, m), 1.78-1.05 (23H, m), 0.98 (3H, s), 0.91-0.88 (3H d, J=6 Hz), 0.86-0.83 (6H, dd, J 1 Hz 6 Hz), 0.66 (3H, s). m/z (ESI) 633.3 (M+H)⁺, 369 (Chol).

### Bocylated Neutral Aminoxylipid (18)

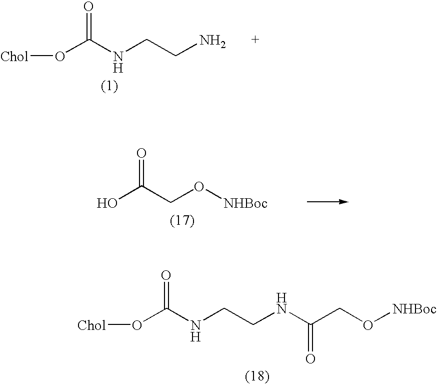

Compound 17 (145 mg, 0.758 mmol) in anhydrous dichloromethane was treated successively with DMAP (292 mg, 2.39 mmol), HBTU (373 mg, 0.987 mmol) and amine 01 (272 mg, 0.576 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography affording pure 18 (302 mg, 81%). ¹H NMR (400 MHz, CDCl₃) 8.56 (s, 1H, BocNHOCH₂), 8.2 (br, CH₂CONHCH₂), 5.5 (m, 1H, Chol C6), 5.4 (m, 1H, Chol-O(CO)NH, 4.5 (m, 1H, Chol C-3), 4.3 (s, 2H, (CO)CH₂ONH₂), 3.4 (m, 2H, O(CO)NHCH₂CH₂), 3.3 (m, 2H, O(CO)NHCH₂CH₂), 2.32 (m, 2H, Chol C-24), 1.46 (s, 3H, Boc), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3 H, J=6.4, Chol C-21), 0.83, 0.82 (2×d, 6H, J=6.5 and 2.0 Hz), 0.68 (s, 3H, Chol C-18); ³C NMR (100 MHz, CDCl₃) 169.6 (NH(CO)CH₂ONH₂), 157.9 (Boc), 156.6 (OCONH), 139.7 (C-5), 122.4 (C-6), 82.8 (Boc), 76.2 ((CO)CH₂ONH₂), 74.4 (C-3), 56.6 (C-14), 56.0 (C-17), 49.9 (C-9), 42.2 (C-13), 40.6 (C4), 39.4-40.6 (C-12, C-4, O(CO)NHCH₂CH₂ overlapping), 38.4 (C-24), 36.9 (C-1), 36.4 (C-10), 36.1 (C-22), 35.7 (C-20), 31.80 (C-8), 321.79 (C-7), 28.1 (C-16 and Boc overlapping), 28.0 (C-2), 27.9 (C-25), 24.2 (C-15), 23.7 (C-23), 22.7 (C-26), 22.5 (C-27), 20.9 (C-11), 19.2 (C-19), 18.6 (C-21) and 11.8 (C-18). ESI-MS 646 [M+H]⁺; HRMS: calculated for C₃₇H₆₄N₃O₆: 646.479512; Found: 646.479874.

### Neutral Aminoxy Lipid (19)

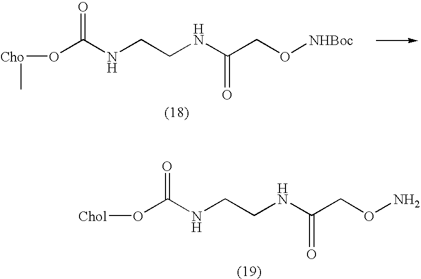

Compound 18 (86 mg, 0.067 mmol) in propan-2-ol (3 ml) then treated with 4M HCl in dioxane (3 ml) and the mixture stirred at room temperature for 4 h. The solvents were removed in vacuo and the residue redissolved in a minimum of 1:5 propan-2-ol:dioxane and the product 19 precipitated with ether as a white solid (28 mg, 84%);. ¹H NMR (400 MHz, d₄-MeOD) 5.35 (m, 1H, Chol C6), 4.8 (m, 1H, Chol-O(CO)NH), 4.5 (s, 2H, (CO)CH₂ONH₂), 4.4 (m, 1H, Chol C-3), 3.3 (m, 2H, O(CO)NHCH₂CH₂), 3.1 (m, 2H, O(CO)NHCH₂CH₂), 2.32 (m, 2H, Chol C-24), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3H, J=6.4, Chol C-21), 0.83, 0.82 (2×d, 6H, J=6.5 and 2.0 Hz), 0.68 (s, 3H, Chol C-18); ³C NMR (100 MHz, CDCl₃) 171.4 (NH(CO)CH₂ONH₂), 158.3 (OCONH), 140.55 (C-5), 123.2 (C-6), 75.4 ((CO)CH₂ONH₂) 71.9 (C-3), 57.5 (C-14), 57.0 (C-17), 51.0 (C-9), 43.0 (C-13), 40.2 (C-4), 40.0-40.6 (C-12, C-4), O(CO)NHCH₂CH₂ overlapping), 39.2 (C-24), 37.8 (C-1), 37.3 (C-10), 36.9 (C-22), 36.6 (C-20), 32.7 (C-8), 32.6 (C-7), 28.9 (C-16), 28.8 (C-2), 28.7 (C-25), 24.9 (C-15), 24.5 (C-23), 23.2 (C-26), 22.9 (C-27), 21.8 (C-11), 19.7 (C-19), 19.2 (C-21) and 12.3 (C-18). ESI-MS 546 [M+H]⁺.

### Cholesterylglycine (20)

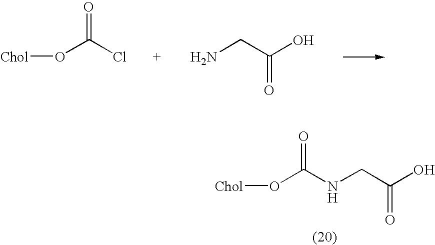

To cholesterol chloroformate (1 g, 2.23 mmol) in dioxane (35 ml) at 0° C. was added NEt₃ (424 μl, 2.23 mmol) and glycine (170 mg, 2.23 mmol) in water (15 ml) and the mixture stirred at r.t. overnight. The reaction was quenched with 7% aqueous citric acid and extracted with dichloromethane. The extracts were dried and concentrated in vacuo affording a residue which was purified by chromatography to afford compound 20 as a white solid (680 mg, 63%); ¹H NMR (400 MHz, CDCl₃) 5.35 (m, 1H, Chol C6), 5.15 (m, 1H. Chol-O(CO)NH), 4.5 (s, 2H, (CO)CH₂ONH₂), 4.5 (m, 1H, Chol C-3), 3.95 (m, 2H, O(CO)NHCH₂), 2.32 (m, 2 H, Chol C-24), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3H, J=6.4, Chol C-21), 0.83, 0.82 (2×d, 6H, J=6.5 and 2.0 Hz), 0.68 (s, 3H, Chol C-18); ³C NMR (100 MHz, CDCl₃) 159.3 (OCONH), 142.4 (C-5), 125.4 (C-6), 75.4 ((CO)CH₂ONH₂) 71.9 (C-3), 57.5 (C-14), 57.0 (C-17), 51.0 (C-9), 43.0 (C-13), 40.040.6 (C-12, C4), 39.2 (C-24), 37.8 (C-1), 37.3 (C-10), 36.9 (C-22), 36.6 (C-20), 32.7 (C-8), 32.6 (C-7), 28.9 (C-16), 28.8 (C-2), 28.7 (C-25), 24.9 (C-15), 24.5 (C-23), 23.2 (C-26), 22.9 (C-27), 21.8 (C-11), 19.7 (C-19), 19.2 (C-21) and 12.3 (C-18). MS-FAB+: 510 [M+Na]⁺.

### Bocylated-cholesteryl-glycyl-hydrazide (22)

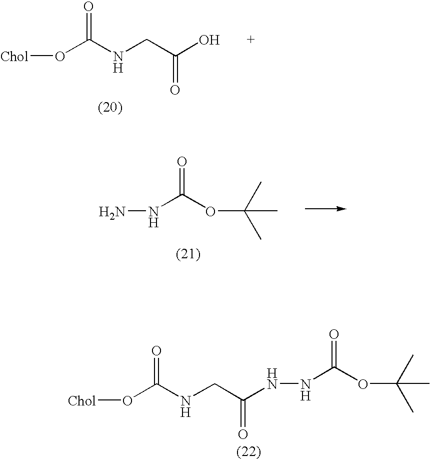

Compound 21 (33 mg, 0.246 mmol) in anhydrous dichloromethane was treated successively with DMAP (73 mg, 0.6 mmol), HBTU (109 mg, 0.287 mmol) and 20 (100 mg, 0.205 mmol) and the mixture stirred at r.t. under a nitrogen atmosphere for 15 h. The reaction was quenched with 7% aqueous citric acid and extracted with dichloromethane. The dried (MgSO₄) extracts were concentrated in vacuo to afford a residue which was purified by flash column chromatography affording pure 22 (103 mg, 83%); ¹H NMR (400 MHz, CDCl₃) 8.6 brs, 1H. BocNH₂NH₂CO), 6.9 (br, CH₂CONH₂NH₂Boc), 5.8 (m, 1H, Chol-O(CO)NH), 5.4 (m, 1H, Chol C6), 4.5 (m, 1H, Chol C-3), 3.9 (s, 2H, (CO)CH₂NH(CO)O), 2.32 (m, 2H, Chol C-24), 1.46 (s, 3 H, Boc), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3H, J=6.4, Chol C-21), 0.83, 0.82 (2×d, 6 H, J=6.5 and 2.0 Hz), 0.68 (s, 3H, Chol C-18); ³C NMR (100 MHz, CDCl₃) 169.7 (BocNH₂NH₂CO), 156.7 (Boc), 155.6 (OCONH), 139.6 (C-5), 122.6 (C-6), 82.0 (Boc), 74.9 (C-3), 56.6 (C-14), 56.2 (C-17), 49.9 (C-9), 42.9 (Gly CH₂), 42.3 (C-13), 39.7 (C-4), 39.4-6 (C-12), 38.4 (C-24), 36.9 (C-1), 36.5 (C-10), 36.2 (C-22), 35.8 (C-20), 31.80 (C-8), 31.79 (C-7), 28.2 (C-16 and Boc overlapping), 28.1 (C-2), 27.9 (C-25), 24.2 (C-15), 23.9 (C-23), 22.8 (C-26), 22.5 (C-27), 21.0 (C-11), 19.3 (C-19), 18.7 (C-21) and 11.8 (C-18). ESI-MS 502 [M+H]⁺, 542 [M+K]⁺; HRMS: calculated for C₃₅H₅₉N₃O₅Na: 624.435242; Found: 624.436356.

### Cholesteryl-glycyl-hydrazide (23)

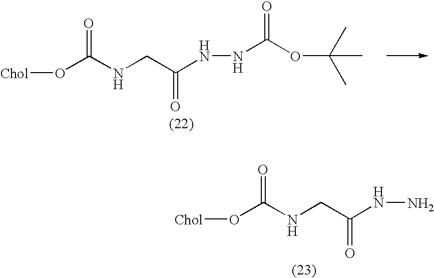

Compound 22 (40 mg, 0.067 mmol) in propan-2-ol (1 ml) then treated with 4M HCl in dioxane (1 ml) and the mixture stirred at room temperature for 30 min. The solvents were removed in vacuo and the residue redissolved in a minimum of 1:5 propan-2-ol:dioxane and the product 23 precipitated with hexanes as a white solid (28 mg, 84%); ¹H NMR (400 MHz, d₄-MeOD) 7.8 (br, CH₂CONH₂NH₂), 5.5 (m, 1H, Chol C6), 4.6 (m, 1H, Chol C-3), 4.0 (s, 2H, (CO)CH₂NH(CO)O), 2.32 (m, 2H, Chol C-24), 1.46 (s, 3H, Boc), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3H, J=6.4, Chol C-21), 0.83, 0.82 (2×d, 6H, J=6.5 and 2.0 Hz), 0.68 (s, 3H, Chol C-18); ¹³C NMR (100 MHz, d₄-MeOD) 169.7 (NH₂NH₂CO), 156.6 (OCONH), 140.3 (C-5), 123.2 (C-6), 75.9 (C-3), 57.4 (C-14), 56.8 (C-17), 50.8 (C-9), 48.4 (gly CH₂), 42.9 (C-13), 40.4 (C-4), 40.1 (C-12), 39.0 (C-24), 37.6 (C-1), 37.2 (C-10), 36.8 (C-22), 36.5 (C-20), 32.5 (C-8), 32.4 (C-7), 28.8 (C-16), 28.7 (C-2), 28.6 (C-25), 24.8 (C-15), 24.4 (C-23), 23.1 (C-26), 22.9 (C-27), 21.7 (C-11), 19.7 (C-19), 19.0 (C-21) and 12.2 (C-18). ESI-MS: 541.7 [M+K]⁺.

### Cholesteryl-carbamate-hydrazide (24)

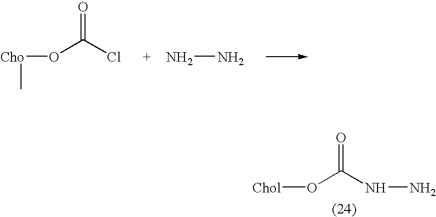

Cholesterol chloroformate (1.0 g, 2.23 mmol) in dichloromethane (90 ml) at 0° C. was added hydrazine hydrate (1 g, 20 mmol) and the reaction slowly warmed to r.t. and stirred overnight. The reaction was quenched with 7% aqueous citric acid and extracted with dichloromethane. The dried (MgSO₄) extracts were concentrated in vacuo to afford a residue which was crystallized from dichloromethane/hexanes affording 24 as a white solid (0.75 g, 76%); ¹H NMR (400 MHz, CDCl₃) 5.4 (m, 1H, Chol C6), 4.55 (m, 1H, Chol C-3), 4.7-3.3 (O(CO)NHNH₂), 2.32 (m, 2H, Chol C-24), 1.46 (s, 3H, Boc), 0.94-2.10 (Chol C-1, 2, 4, 7, 8, 9, 11, 12, 14, 15, 16, 17, 20, 22, 23, 25), 1.0 (s, 3H, Chol C-19), 0.89 (d, 3H, J=6.4, Chol C-21), 0.83, 0.82 (2×d, 6H, J=6.5 and 2.0 Hz), 0.68 (s, 3H, Chol C-18); ¹³C NMR (100 MHz, CDCl₃) 158.3 (OCONH), 139.5 (C-5), 122.7 (C-6), 75.2 (C-3), 56.6 (C-14), 56.1 (C-17), 49.9 (C-9), 42.2 (C-13), 39.7 (C-4), 39.4 (C-12), 38.4 (C-24), 36.9 (C-1), 36.5 (C-1-0), 36.1 (C-22), 35.7 (C-20), 31.8 (C-8), 31.77 (C-7), 28.2 (C-25), 28.0 (C-16), 27.9 (C-2), 24.2 (C-15), 23.8(C-23), 22.8 (C-26), 22.5 (C-27), 21.0 (C-11), 19.2(C-19), 18.6 (C-21) and 11.8 (C-18). ESI-MS: 484.63 [M+K]⁺.

### (Boc)aminooxylipid(25)

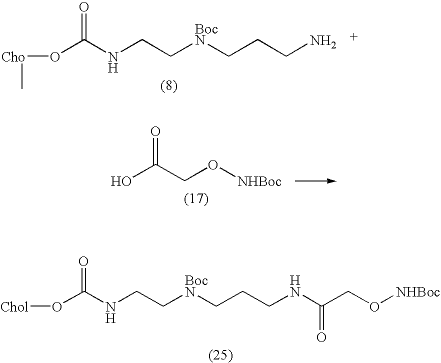

N-hydroxysuccinimide (0.36 g, 3.13 mmol, 1 equiv), 17 (0.6 g, 3.13 mmol, 1 equiv), and N,N′-dicyclohexylcarbodiimide (0.68 g, 3.13 mmol, 1 equiv) were dissolved in EtOAc (90 mL), and the heterogeneous mixture was allowed to stir at room temperature overnight. The mixture was then filtered through a pad of Celite® to remove the dicyclohexylurea, which was formed as a white precipitate (rinsed with 60 mL of EtOAc), and added to a solution of 8 (1.97 g, 3.13 mmol, 1 equiv) in THF (10 mL). A pH of 8 was maintained for this heterogeneous reaction by addition of triethylamine (6 mL). The resulting mixture was allowed to stir at room temperature overnight. On completion the mixture was filtered and the solvent was removed under reduced pressure to give after purification by flash-chromatography (CH₂Cl₂/MeOH/NH₃92:7:1) 25 as a white solid. Yield (2.3 g, 90%); ¹H NMR (270 MHz, CDCl₃): δ=5.33-5.35 (m, 1H, H6′), 4.4-4.52 (m, 1H, H3′), 4.3 (s, 2H, H90, 3.2-3.42 (m, 8H, H1, H2, H4, H6), 2.23-2.35 (m, 2H, H4′), 1.7-2.1 (m, 7H, H2′, H7′, H8′, H5), 1.44-1.46 (m, 18H, 2 Boc), 1-1.73 (m, 21H, H1′, H9′, H11′, H12′, H14′-H17′, H22′-H25′), 0.98 (3H, s, H-19′), 0.85 (d, J=6.5 Hz, 3H, H21′), 0.83 (d, J=6.5 Hz, 6H, H26′&H27′) and 0.65 (s, 3H, H18′); MS (FAB⁺): m/z=803 [M+H]⁺, 703 [M-Boc]⁺, 647, 603 [M-2Boc]⁺, 369, 279, 255, 235, 204, 145, 95, 69.

### Charged Aminoxylipid (1)

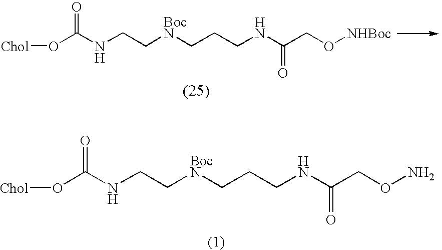

To a solution 25 (1.1 g, 1.36 mmol, 1 equiv) in CH₂Cl₂ (10 mL) was added TFA (2 mL, 20.4 mmol, 15 equiv) at 0° C. The solution was allowed to stir at room temperature for 5 hours. On completion toluene was added to azeotrope TFA from the reaction mixture. The solvents were removed in vacuo to afford after purification by chromatography (CH₂Cl₂/MeOH/NH₃92:7:1 to 75:22:3) 1 as a white solid (709 mg, Yield: 86%); IR (CHCl₃): νₘₐₓ=3306, 2948, 2850, 2246, 1698, 1647, 1541, 1467, 1253, 1133; ¹H NMR (270 MHz, CDCl₃): δ=5.26-5.4 (m, 1H, H6′), 4.44.52 (m, 1H, H3′), 4.12 (s, 2H, H9), 3.34-3.41 (m, 2H, H2), 3.15-3.3 (m, 2H, H4), 2.6-2.74 (m, 4H, H1 & H6), 2.14-2.39 (m, 2H, H4′), 1.62-2.1 (m, 7H, H2′, H7′, H8′, H5), 1.02-1.6 (m, 21H, H1′, H9′, H11′, H12′, H14′-H17′, H22′-H25′), 0.96 (3H, s, H-19′), 0.86 (d, J=6.5 Hz, 3H, H21′), 0.83 (d, J=6.5 Hz, 6H, H26′&H27′) and 0.66 (s, 3H, H18′); MS (FAB⁺): m/z=603 [M+H]⁺, 369[Chol]⁺, 160, 137, 109, 95, 81, 69, 55.

### Stability of Aminoxy-Lipid 1 Containing Liposomes and Lipoplexes

(A) Studies on LMD Systems Devoid of Aminoxy-Lipid 1

LMD composed of DOPE:lipidB198 (60:40, molar ratios) liposomes at the standard formulation ratio 12:0.6:1 were subjected to a stability analyses. LMDs were incubated with different amounts of PEG²⁰⁰⁰-dialdehyde for 16 hours in HEPES 4 mM (pH 7). Subsequently, samples were added into OptiMEM and the respective sizes measured by PCS over 20 minutes (FIG. 2). A clear effect of stabilization was observed for increasing amounts of PEG²⁰⁰⁰-dialdehyde. This stabilization suggests the formation of a Schiff-base, thus stabilizing the particle by the formation of a covalent C═N bond between surface exposed amines of the lipoplex (DOPE, lipidB198) and the aldehyde from the PEG. In order to rule out non-specific absorption of polyethylene glycol to the LMD surface, control experiments were carried out with PEG derivatives containing a thiol, an amine or two amine functions, respectively (FIG. 3). The results clearly suggest a specific interaction of the aldehyde-containing PEG with the aminoxy functionality, whereas the other functionalised PEG derivatives exhibit very weak, non-specific effects. In order to verify the suggested formation of a Schiff-base, we turned to a LMD formulation where 10% of lipidB198 was replaced by aminoxy-lipid 1.

Biochemicals and Chemicals:

Dioleoylphosphatidyl-ethanolamine (DOPE) was purchased from Avanti Lipid (Alabaster, Ala., USA). Plasmid nis-pCMV βGalactosidase was produced by Bayou Biolabs (Harahan, La., USA). Lipid-B198 were synthesised in our Laboratory. Mu-peptide was synthesised by standard Fmoc based Merrifield solid phase peptide chemistry on Wang resin.

Synthesis:

Preparation of Liposomes:

Liposomes were prepared as follows. The adequate lipid mixture in dichloromethane was dried as a thin layer in a 100 ml round-bottomed flask that was further dried under vacuum for 2 h. The lipid film was hydrated in 4 mM Hepes (pH 7) to give a final concentration of 5 mg/ml lipid. Preparation of small unilamelar vesicles by extrusion was performed after brief sonication by extruding ten times the suspension through two stacked polycarbonate filters (0.1 μm pore, Osmonids) using Extruder (Lipex Biomembranes) under Nitrogen. Lipid concentration of the extruded liposomes was determined by Steward assay.

Preparation of MD and LMD and LD

Preparation of LD (lipid:DNA) and LMD (Lipid:Mu:DNA) complexes: DNA stock solution (typically 1.2 mg/ml) was added to a vortex mixing diluted solution of Mu in distilled Water at 0.6 weight ratio to obtain a 0.2 mg/ml DNA final concentration. The MD solution was then slowly added to the liposomes under vortex at a weight ratio DNA:Lipids of 1:12. Sucrose diluted in 4 mM Hepes is finally added to obtain an LMD preparation at the desired DNA concentration in 4 mM Hepes, 6% sucrose. A DNA solution of 0.2 mg/ml was slowly added to the liposomes under vortex at a weight ratio of DNA:Lipids of 1:12. Sucrose diluted in HEPES 4 mM pH 7 is finally added to obtain an LD preparation at the desired DNA concentration in HEPES 4 mM (pH 7), 6% sucrose.

Stability Study on LMD systems Containing LiposomesB198:DOPE (50:50)

LMD composed of DOPE:lipidB198 (60:40, molar ratios) liposomes at 0.15 mg/ml (DNA concentration) were subjected to stability analyses in OptiMEM. LMDs were incubated with different amounts of PEG²⁰⁰⁰-dialdehyde for 16 hours/4° C. in HEPES 4 mM (pH 7) and the final concentration adjusted at 0.1 mg/ml. Subsequently, samples were added into OptiMEM and the respective sizes measured using dynamic light scattering technique on a Photon Correlation Spectrometer (N4 plus, Coulter). The parameters used were: 20° C., 0.0890 cP (viscosity), reflex index of 1.33, angle 90°, 632.8 nm (wavelength). A clear effect of stabilization was observed for increasing amounts of PEG²⁰⁰⁰-dialdehyde.

This stabilization suggests the formation of a Schiff-base, thus stabilizing the particle by the formation of a covalent C═N bond between surface exposed amines of the lipoplex (DOPE, lipidB198) and the aldehyde from the PEG. In order to rule out non-specific absorption of polyethylene glycol to the LMD surface, control experiments were carried out with PEG derivatives containing a thiol, an amine or two amine functions, respectively (FIG. 3). The results clearly suggest a specific interaction of the aldehyde-containing PEG with the amine functionality, whereas the other functionalized PEG derivatives exhibit very weak, non-specific effects.

LMD composed of DOPE:lipidB198 (50:50, molar ratios) liposomes at 0.15 mg/ml (DNA concentration) were subjected to stability analyses in serum. LMDs were incubated with different amounts of PEG²⁰⁰⁰-dialdehyde for 16 hours/4° C. in HEPES 4 mM (pH 7) and the final concentration adjusted at 0.1 mg/ml. Subsequently, 60 μl of LMD of different composition were mixed with 240 μl of serum and the mixtures were incubated at 37° C. with gentle shaking. The absorbance at 600 nm was then recorded on an Ultrospec 4000 spectrophotometer (Phamarcia Biotech Ltd, Cambridge, England) at different times with serum alone as blank reference. No significant stabilization effect was observed for increasing amounts of PEG²⁰⁰⁰-dialdehyde (FIG. 7). LD composed of DOPE:lipidB198:Cholesterol (45:30:25, molar ratios) liposomes at 0.1 mg/ml (DNA concentration) were subjected to analyses in serum. LDs were incubated with different molar percent (versus total molar lipid content) of PEG2000-dialdehyde, OpF-acon-PEG3400-mal, NHS-PEG3000-mal for 16 hours/4° C. in HEPES 4 mM (pH 7) and the final concentration adjusted at 0.09 mg/ml. Subsequently, 16.6 μl of LD of different composition were mixed with 50 μl of serum and the mixtures were incubated at 37° C. 5 μl of LD was sampled at different time points to measure the size of the resulting particle on a Photon Correlation Spectrometer (sample were diluted in HEPES 4 mM pH7 for measurement). FIG. 12.

This suggest that the formed Schiff-base between the exposed amines of the lipoplexes (DOPE, B198) and PEG-dialdehyde is not highly stable in serum. The effect of this PEG is weak on an unstable formulation like LMD (B198:DOPE) (FIG. 7) and more noticeable on a more stable formulation like LD (DOPE, B198, cholesterol).

FIG. 12 suggest that the pH sensitive Opf-acon-PEG-Mal is actively coupling on the amine of the lipoplexes and do produce a very strong stabilisation effect.

Studies with Serinelipid 13 Containing Liposomes

DOPE:Serinelipid 13 (50:50) liposomes were used to form LMD vectors at standard 12:0.6:1 ratios (liposome:mu:pDNA) and stability profile established in presence of different amounts of PEG ²⁰⁰⁰-dialdehyde. The complex was allowed to equilibrate for 16 hours in HEPES 4 mM (pH 7) before adding samples into OptiMEM. A clear relationship between the amount of PEG present with the LMD and its complex stability could be established. LMDs without added PEG very rapidly increase in size, whereas addition of 20% PEG (mass ratio, corresponds approximately to 6% molar with respect to the liposomes) increased slowly in size (FIG. 8). The proof of specific formation of a covalent bond between the lipid and the PEG-dialdehyde comes from a comparison with thiolated PEG. Only the aldehyde containing PEG affords stable LMDs, whereas the other PEG exhibit no stabilization pattern. Together, these experiments suggest the formation of a covalent, Schiff-base-like linkage between the polyethylene glycol and surface amine groups of the serine cholesterol based compound.

LMD composed of DOPE:Serinelipid 13 (50:50) liposomes at the standard formulation ratio 12:0.6:1 were subjected to stability analyses in serum. LMDs were incubated with different amount of PEG²⁰⁰⁰-dialdehyde for 20 hours in HEPES 4 mM (pH 7). Subsequently, 60 μl of LMD of different composition at 100 μg/ml were mixed with 240 μl of serum and the mixtures were incubated at 37° C. with gentle shaking. The absorbance at 600 nm was then recorded at different times with serum alone as blank reference. Significant stabilization effect was observed (FIG. 9) for increasing amounts of PEG²⁰⁰⁰-dialdehyde.

This suggests that the formed Schiff-base between surface exposed serine and PEG-aldehydes of the LMD is stable enough to reduce serum-induced aggregation.

Transfection Experiments:

Transfection Protocols on Panc-1 Cells in OptiMEM and Serum (90%)

General. Cultured Panc-1 or OVCAR-1 cells were seeded at 2E5 cells per well in 48-well culture plates and grown to approximately 70% confluence in DMEM at 37° C. and 5% CO₂. The cells were washed in PBS before the transfection media was administered to each well (0.250 ml of serum or OptiMem). 0.5 μg of LMD (DNA) was added to each well for 1 hour. Cells were then rinsed 3 times with PBS and left for 24 hours to grow in normal medium (NGM). Cells were scraped from the plates and β-Gal expression was assayed by using the chemiluminescent Reporter Gene Assay Kit of Roche Diagnostics.

Results. Transfection results demonstrate that with increasing amount of PEG-bisaldehyde, decreased activity is observed. This is consistent with a covalent coupling (Schiff-base formation) of the PEG to the LMD, which is further underlined by the PEG-SH control which did not affect transfection levels. The decrease in transfection can either be due to a decreased cellular uptake of the vectors due to the PEG attachment to the LMD surface, thus shielding positive charges, or, alternatively, by an inhibitory intracellular effect of the PEG.

Conclusions

The surface reaction of an aldehyde/ketone-functionalised PEG with (a) an amine or (b) a serine-containing lipid (e.g. Serinelipid 13) is achieved. The resulting bond is very labile (a) or more stable (b). In both cases, the only side product formed in the course of the condensation reaction is water. Therefore, this method represents an extremely powerful and elegant way to stabilize drug or gene delivery systems that retain part of their transfection activity (FIGS. 10 and 11) and exhibit a strong stabilization profile. It is expected that this concept is ideal for balancing between stabilization and functionality of drug/gene delivery vectors. Furthermore, this concept allows for the facile one-pot reaction of drug/gene delivery vector with the bifunctional stealth compound and a thiol containing targeting ligand.

Post-Coupling, Serum Stabilization, Triggerability and In Vitro Transfection

Profiles

General Remarks. Each of the triggerable lipids listed in table 1 was formulated into liposomes as a third lipid beside LIPIDB198 and DOPE at optimised ratios (see figures). The liposomes were extruded through 100 nm membranes (10×) and sized by PCS. LD (liposome+pDNA) were produced by slow addition of a diluted solution of pDNA in HEPES (4 mM) to give a final concentration of 0.1 mg pDNA/mL. LDs were stored in presence of 6% sucrose at 4° C. if not immediately used for transfection. Three formulations were found to be particularly interesting, which were LipidB198/DOPE/cholesterol (45:30:25), LipidB198/DOPE/lipid 23, and LipidB198/DOPE/aminoxylipid 1.

LIPIDB198/DOPE/Cholesterol

Serum Stability

LDs composed of DOPE:LipidB198:cholesterol (45:30:25, molar ratios) liposomes at 0.1 mg/ml (pDNA) were analyzed after subjection to serum. LDs were incubated with different molar percentages (versus total molar lipid content) of PEG²⁰⁰⁰-dialdehyde, OpF-acon-PEG³⁴⁰⁰-mal, NHS-PEG³⁴⁰⁰-mal for 16 h/4° C. in HEPES 4 mM (pH 7). The final concentration was adjusted at 0.09 mg/ml. Subsequently, 16.61 μl of LD of different composition were mixed with 50 μl of serum and the mixtures were incubated at 37° C. five μl of LD was sampled at different time points to measure the size of the resulting particle by PCS (each sample was diluted in HEPES 4 mM pH7 for the measurement).

Conclusion

The results suggest that Schiff-base formed between the exposed amines of the lipoplexes (DOPE, LipidB198) and PEG-dialdehyde is not very stable in serum. The effect of this PEG is weak on an unstable formulation like LMD (LipidB198/DOPE) (FIG. 7) and more noticeable on a more stable formulation like LD (DOPE/LipidB198/cholesterol) (FIG. 12).

FIG. 12 suggests that the pH sensitive OpF-acon-PEG-mal is actively coupling on the amine of the lipoplexes yielding a very strong stabilization effect.

LIPIDB198/DOPE/Lipid 23

Serum Stability

LDs composed of DOPE:lipidB198:lipid 23 (45:30:25, m/m/m) liposomes at 0.1 mg/ml (pDNA) were analysed after exposure to serum. LDs were incubated with different molar percentages (versus total molar lipid content) of PEG²⁰⁰⁰-dialdehyde, OpF-acon-PEG³⁴⁰⁰-mal and PEG⁶⁰⁰⁰-SH for 16 h/4° C. in HEPES 4 mM (pH 7) and the final concentration adjusted at 0.09 mg/ml. Subsequently, 16.6 μl of LD of different composition were mixed with 50 μl serum and the mixtures incubated at 37° C. Five μl of LD was sampled at different time points and the size was measured by PCS (sample were diluted in HEPES 4 mM pH7 for measurement).

pH Release

LDs composed of DOPE:LipidB198:lipid 23 (45:30:25, molar ratios) liposomes at 0.1 mg/ml (pDNA) were subjected to stability analyses in serum after pH 5.3 exposure. LDs were incubated with different molar percentages (versus total molar lipid content) of PEG²⁰⁰⁰-dialdehyde or OpF-acon-PEG³⁴⁰⁰-mal for 16 h/4° C. in HEPES 4 mM (pH 7) and the final concentration adjusted at 0.09 mg/ml. Prior to serum stability experiment (similar as previous), LDs were incubated 3 h at pH5.3 by addition of HCl.

Transfection

LDs composed of DOPE:lipidB198:lipid 23 (45:30:25, molar ratios) liposomes at 0.1 mg/ml (DNA concentration) were transfected on OVCAR-1 cells following the described transfection protocol.

Targeting

LD composed of DOPE:LipidB198:lipid 23 (45:30:25, molar ratios) liposomes (ratio pDNA:lipid=1:14) at 0.1 mg/ml (pDNA) were subjected to targeting experiments. Firstly a solution of OpF-acon-PEG³⁴⁰⁰-mal was incubated 1 h at pH 8 with a solution of folate-cysteine peptide to give OpF-acon-PEG³⁴⁰⁰-cys-folate which subsequently was added to the LD solution (1 or 10 molar % versus total molar lipid content). Control LDs were produced by submitting an OpF-acon-PEG³⁴⁰⁰-mal solution to the same treatment without addition of the targeting peptide.

The mix was left to incubate for 16 h/4° C. in HEPES 4 mM (pH 7) and dialyzed (MCO=10000) 24 h against the same buffer to obtain a 40 μg/ml targeted LD solution. Subsequently, 37.5 μl of LD of different composition were mixed with 50 μl of serum and the mixtures were incubated at 37° C. Eight microliters LD were sampled at different time points to measure the size of the resulting particle by PCS (sample were diluted in HEPES 4 mM pH7 for measurement).

These LD were transfected on OVCAR-1 cells following the described transfection protocol.

Conclusion

FIG. 13 demonstrates the high stability of LD containing the neutral hydrazide lipid 23. This suggests that the carboxylic hydrazone adduct formed between the hydrazide of the lipoplexes and PEG²⁰⁰⁰-dialdehyde is highly stable in serum. The control experiment using PEG⁶⁰⁰⁰-SH clearly demonstrate that this effect is due to the aldehyde function forming a serum stable adduct.

FIG. 13 suggests that the pH sensitive OpF-acon-PEG³⁴⁰⁰-mal is strongly coupling to the hydrazine lipid 23, resulting in a highly serum resistant lipoplex formulation.

FIG. 14 demonstrates that in the condition of the assay the acon-PEG³⁴⁰⁰-mal coupled LD (containing lipid 23) and the non-modified LD are not influenced by the pH incubation (similar results as FIG. 13). The pH sensitive hydrazone adduct is strongly influenced by the pH (5.3) resulting in a much less stable particle than in FIG. 13. FIG. 19 demonstrates that the stable LD containing hydrazide lipid 23. does transfect even in 95% containing media. The decrease of transfection observed with increasing amount of PEG is consistent with a covalent coupling of the PEG on the LD. This could be due to a decrease of the cellular uptake of the vectors due to PEG attachment or an inhibitory intracellular effect of PEG.

FIG. 20 demonstrates the efficient coupling of both OpF-acon-PEG³⁴⁰⁰-mal and OpF-acon-PEG³⁴⁰⁰-cys-folate onto the LD. This LD is highly stable when modified with 10 molar % OpF-acon-PEG³⁴⁰⁰-mal or 10 molar percentage of OpF-acon-PEG³⁴⁰⁰-cys-folate. FIG. 22 demonstrates the targeting potential ability of the post-modified LD system. When sufficient targeting moiety is coupled to the lipoplexes (10 molar percentage) a clear increase (3 folds in 10% serum and 6 folds in 95% serum) due to targeting of the folate receptor of the OVCAR-1 cell line is observed. The transfection level of the 10% OpF-acon-PEG³⁴⁰⁰-cys-folate LD in 95% serum is equivalent to the one of the unmodified particle in the same condition.

Summary. Altogether these results suggest that the hydrazide lipid 23 coupled to the aldehyde of the PEG-dialdehyde resulting in a pH sensitive but serum resistant conjugate. The PEG containing a cis-aconityl bond did not yield a pH release under the conditions of the assay but is expected to be pH sensitive in the more challenging in vitro/in vivo condition ¹³⁻¹⁵.

The in vitro transfection results demonstrate that the resulting particle is able to transfect even under very challenging conditions like 95% serum. The stability of this particle combined with its pH release potential and its transfection ability are considered to be ideal for systemic applications.

The resulting lipoplex can be targeted using the folate receptor. This particle is highly stable and does transfect more efficiently than the one without the targeting moiety.

LIPIDB198/DOPE/aminoxylipid 1

Serum Stability

LD composed of DOPE:LipidB198:aminoxylipid 1 (45:30:25, molar ratios) liposomes at 0.1 mg/ml (pDNA) were analysed after exposure to serum. LDs were incubated with different molar percentages (versus total molar lipid content) of PEG²⁰⁰⁰-dialdehyde, OpF-acon-PEG³⁴⁰⁰-mal and PEG⁶⁰⁰⁰SH for 16 h/4° C. in HEPES 4 mM (pH 7) and the final concentration adjusted at 0.09 mg/ml. Subsequently, 16.6, μl of LD of different composition were mixed with 50 μl of serum and the mixtures were incubated at 37° C. Five microliters of LD were sampled at different time points to measure the size of the resulting particle by PCS (sample were diluted in HEPES 4 mM pH7 for measurement).

pH Release

LD composed of DOPE:LipidB198: aminoxylipid 1 (45:30:25, molar ratios) liposomes at 0.1 mg/ml (DNA concentration) were subjected to stability analyses in serum after pH 5.3 exposure. LDs were incubated with different molar percentages (versus total molar lipid content) of PEG²⁰⁰⁰-dialdehyde or OpF-acon-PEG³⁴⁰⁰-mal for 16 h/4° C. in HEPES 4 mM (pH 7) and the final concentration adjusted at 0.09 mg/ml. Prior to serum stability experiment (similar as previous), LDs were incubated 3 h at pH 5.3 by addition of HCl.

Transfection

LD composed of DOPE:lipidB198:aminoxylipid 1 (45:30:25, molar ratios) liposomes at 0.1 mg/ml (pDNA) were transfected on OVCAR-1 cells following the described transfection protocol.

Targeting

LD composed of DOPE:lipidLipidB198:aminoxylipid 1 (45:30:25, m/m/m) liposomes (pDNA:lipid 1:12, w/w) at 0.1 mg/ml (DNA concentration) were subjected to targeting experiments. Firstly a solution of OpF-acon-PEG³⁴⁰⁰-mal was incubated 1 hour at pH 8 with a solution of folate-cysteine peptide to afford OpF-acon-PEG³⁴⁰⁰-cys-folate which was added to the LD solution (1 or 10 molar % versus total molar lipid content). Control LDs were produced by submitting an OpF-acon-PEG³⁴⁰⁰-mal solution to the same treatment without addition of the targeting peptide.

The mix was left to incubate for 16 h/4° C. in HEPES 4 mM (pH 7) and dialysed 24 h against the same buffer to obtain a 40 μg/ml targeted LD solution.

Subsequently, 37.5 μl of LD of different composition were mixed with 50 μl of serum and the mixtures were incubated at 37° C. 8 μl of LD was sampled at different time points to measure the size of the resulting particle by PCS (samples were diluted in HEPES 4 mM pH7 for measurement).

These LD were then transfected on OVCAR-1 cells following the described transfection protocol.

Conclusion

FIG. 15 suggests that the conjugation between the aminoxylipid 1 of the lipoplexes and PEG²⁰⁰⁰-dialdehyde is highly stable in serum. A control experiment using PEG⁶⁰⁰⁰-SH did not yield any such effect.

FIG. 15 suggests that the pH sensitive OpF-acon-PEG³⁴⁰⁰-mal strongly couples to the aminoxylipid 1 of the lipoplexes producing a very strong stabilization effect.

FIG. 16 demonstrates that in the condition of the assay the acon-PEG³⁴⁰⁰-mal and PEG²⁰⁰⁰dialdehyde coupled LDs and the non-modified LD are not influenced by the pH incubation (similar results as FIG. 15).

FIG. 18 demonstrates the superiority in 95% serum of LD containing the aminoxylipid 1 (LD composed of LipidB198:DOPE hardly transfect in 95% serum). The decrease in transfection observed with increasing amount of PEG is consistent with a covalent coupling of the PEG on the LD. This could be due to a decrease of the cellular uptake of the vectors due to PEG attachment or an inhibitory intracellular effect of PEG.

FIG. 21 demonstrates the efficient coupling of both OpF-acon-PEG³⁴00-mal and OpF-acon-PEG³⁴⁰⁰-cys-folate onto the LD. This LD is more stable when modified with 10 molar % OpF-acon-PEG³⁴⁰⁰-mal or 10 molar percentage of OpF-acon-PEG³⁴⁰⁰-cys-Folate. FIG. 23 demonstrates the targeting potential ability of the post-modified LD system. When sufficient targeting moiety is coupled to the lipoplexes (10 molar percentage) a clear increase (3.6 folds in 10% serum and 7.2 folds in 95% serum) due to targeting of the folate receptor of the OVCAR-1 cell line is observed.

Summary. Altogether these results suggest that the aminoxylipid 1 coupled to the aldehyde of the PEG²⁰⁰⁰-dialdehyde does not result in a pH sensitive conjugate. The PEG containing a cis-aconityl bond did not demonstrated pH release in the condition of the assay but is expected to be pH sensitive in the more challenging in vitro/in vivo condition¹⁶. The in vitro transfection results demonstrate that the resulting particle is able to transfect very efficiently even in very challenging condition like 95% serum. The resulting lipoplex can be targeted using the folate receptor. This particle is more stable in 95% serum and do transfect far more efficiently than the one without the targeting moiety.

Study with Lipid 14, 16, 24 Containing Liposome Formulations

Serum Stability

LDs composed of DOPE:LipidB198:lipid 14; DOPE:LipidB198:lipid 16; (45:30:25, molar ratios) liposomes at 0.13 mg/ml (pDNA) were analyzed after subjection to serum. LDs were incubated with different molar percentages (versus total molar lipid content) of PEG²⁰⁰⁰-dialdehyde for 16 h/4° C. in HEPES 4 mM (pH 7). The final concentration was adjusted at 0.1 mg/ml. Subsequently, 60 μl of LD of different composition were mixed with 240 μl of serum and the mixtures were incubated at 37° C. The absorbance at 600 nm was then recorded at different time (turbidity).

Transfection

LD composed of DOPE:LipidB198:lipid 14, DOPE:LipidB198:lipid 16; DOPE:LipidB198:lipid 2.4 DOPE:LipidB198:LipidB198, DOPE:LipidB198:cholesterol and DOPE:LipidB198:aminoxy-lipid 1 (45:30:25 molar ratios) were modified with different molar percentage of PEG²⁰⁰⁰-dialdehyde.

These LDs were transfected on Panc-1 cells following the described transfection protocol.

Conclusion

FIG. 24a suggest that the conjugate formed between the exposed cysteines of the lipoplexes containing lipid 14 and PEG-dialdehyde is not very stable in serum. The effect of dialdehyde PEG on this inherently unstable formulation is weak and only noticeable at high ratios of PEG (25 molar %).

FIG. 24b suggest that the conjugate formed between the exposed cysteines of the lipoplexes containing lipid 16 and PEG-dialdehyde is stable in serum. The effect of this PEG is noticeable.

FIG. 17 demonstrates the decrease in transfection (in 10% containing medium) observed with increasing amount of PEG that is consistent with a covalent coupling of the PEG on the LDs. This could be due to a decrease of the cellular uptake of the vectors due to PEG attachment or an inhibitory intracellular effect of PEG.

Summary. Altogether these results suggest that the cysteine-containing lipid 16 and 14 couple onto the aldehyde of the PEG²⁰⁰⁰-dialdehyde. The resulting complexes are more stable in serum and do express low transfection level on Panc-1 in 10% serum containing medium when coupled with high molar percent of PEGs. LDs containing lipid 24 are able to transfect in growth medium.

Biological Evaluation II: Ex Vivo Transfecion Studies

General

Hippocampal slices were prepared from Wistar rats as described in detail underneath, and incubated with three different type of lipoplexes which differed in their liposome composition. Formulation I: lipoplex with LIPIDB198/DOPE (50:50, m/m); formulation II: lipoplex with LIPIDB198/DOPE/aminoxylipid 1 (30:60:10, m/m/m); formulation III: lipoplex with LIPIDB198/DOPE/aminoxylipid 1 (30:60:10, m/m/m) incubated with dialdehyde²⁰⁰⁰ (10%).

Preparation of Hippocampal Slices

This study was carried out on 27-21 day old Wistar rats (WAG/GSto, Moscow, Russia). After rapid decapitation, rat brains were immediately transferred to a Petri dish with chilled (4° C.) solution of the following composition: 120 mM NaCl, 5 mM KCl, 26 mM NaHCO₃, 2 mM MgCl₂ and 20 mM glucose (solution 1). Calcium salts were omitted to reduce possible neuronal damage. The solution was constantly oxygenated with 95%O₂/5% CO₂ gas mixture to maintain pH=7.4. Hippocampal slices (300-400 μm thick) were cut manually with a razor blade along the alveolar fibres to preserve the lamellar structure of excitatory connections. During the preincubation, the slices were kept fully submerged in the extracellular solution: 135 mM NaCl, 5 mM KCl, 26 mM NaHCO₃, 1.5 mM CaCl₂, 1.5 mM MgCl₂, 20 mM glucose (solution2) (pH=7.4, bubbled with 95% O₂/5% CO₂) at 30-31° C. Experiments were conducted in extracellular solution of following composition: 150 mM NaCl, 5 mM KCl, 20 mM HEPES, 2 mM CaCl₂, 1 mM MgCl₂, 10 mM glucose (solution 3) (pH=7.4, no oxygenation). During incubation with lipoplexes, slices were kept for 1 hour in:
1. case: in solution 2 oxygenated in advance but not during loading procedure. After incubation slices were kept in solution 2 with oxygenation for 8 hours.
2. case: in solution 3 (aminoacids and serums) without oxygenation. Lipoplexes were not removed from extracellular solution. Slices were kept in CO₂ incubator at 37° C. for more than 24 hours.
   Results

FIG. 25 Picture 1 and 3 show a microglial cell on the surface of a slice after transfection with formulation 11 consisting of the liposome formulation LIPIDB198/DOPE/aminoxylipid 1 (30:60:10, m/m/m). It appears that the lipoplex is trapped by phagocytosis. Picture 2 shows pyramidal neurons from the CA1 zone of the hippocampus after transfection with the formulation II. Picture 4 shows a layer of pyramidal neurons (low magnification) after transfection with formulation III.

Conclusion

Post-coated sample III shows the significant tissue intrusion (endocytosis) with an average of 120-140 μm, as detected by fluorescence microscopy, showing a shallow widespread fluorescence underneath the surface investigated. Samples one and two were phagocytosed while exposed to the surface.

Biological Evaluation III: In Vivo Transfecion Studies

General

Female MF-1 mice (35 g) were anaesthetised with 200 μl ketamin:rompun (2:1 v/v) and administered a series of different lipoplex constructs at 10 μg, 20 μg or 30 μg pDNA per animal in a total volume of 30 μl PBS by intranasal installation. All lipoplex samples were prepared at a pDNA concentration of 0.1 mg/mL in HEPES, 4 mM (pH 7), with a final concentration of 10% sucrose, total pDNA 100 μg. Each sample was incubated for 72 hours at 4° C. with dialdehyde²⁰⁰⁰ before concentrating on a vacuum rotavap to a final pDNA concentration of 1.0 mg/mL (i.e. the total final volume being 100 μL). For a better control of formulation, the pDNA component was precondensed with either the adenoviral core peptide mu or C₁₈-mu.

Samples

Standard LMD(a)

Condensing species: μ, 0.6 mass equivalents
Plasmid:pNGVL-1 (β-galactosidase, 7.5 kb), 1 equivalent
Liposomes: B198/DOPE, 12 mass equivalents.
   LMD(AO)(b)
Condensing species: μ, 0.6 mass equivalents
Plasmid:pNGVL-1 (β-galactosidase, 7.5 kb), 1 equivalent
Liposomes: B198/DOPE/aminoxy lipid 1 (30:60:10, m/m/m), 12 mass equivalents.
   LMD(AO/PEG-aldehyde)(c)
Condensing species: μ, 0.6 mass equivalents
Plasmid:pNGVL-1 (β-galactosidase, 7.5 kb), 1 equivalent
Liposomes: B198/DOPE/A01 (30:60:10, m/m/m), 12 mass equivalents
0.5% PEG²⁰⁰⁰-dialdehyde.
   LMD18(AO)(d)
Condensing species: C18-μ, 0.6 mass equivalents
Plasmid:pNGVL-1 (β-Galactosidase, 7.5 kb), 1 equivalent
Liposomes: B198/DOPE/AO1 (30:60:10, m/m/m), 12 mass equivalents.
   LMD18(AO/PEG-aldehyde)(e)
Condensing species: C18-μ, 0.6 mass equivalents
Plasmid:pNGVL-1 (β-galactosidase, 7.5 kb), 1 equivalent
Liposomes: B198/DOPE/AO1 (30:60:10, m/m/m), 12 mass equivalents.
   Procedure

Female MF-1 mice (35 g) were anaesthetised with 200 μl ketamin:rompun (2:1 v/v) and administered LMD constructs 10 μg, 20 μg or 30 μg per animal in a total volume of 30 μl PBS by intranasal installation. After 48 h animals were killed and the trachea and lungs excised. Tissues were homogenised in 1 ml lysis buffer and β-gal expression determined by ELISA using a commercially available assay kit (Boehringer Mannheim). Levels of β-gal were standardised to the protein content of each sample, which was determined using the bicinconinic acid (BCA) protein assay system (Pierce).

FIG. 26. In vivo efficacy of samples LMDa-e at 10, 20 and 30 μg/animal pDNA intranasal administration. Plasmid NGVL-1 (7.5 kb μ-gal). A, μ/B198/DOPE; B μ/B198/DOPE/AO1; C, μ/B198/DOPE/AO1+5% PEG²⁰⁰⁰-dialdehyde; D, C18-μ/B198/DOPE/AO1; E, C18-μ/B198/DOPE/AO1+5% PEG²⁰⁰⁰-dialdehyde.

Results and Conclusion

The dialdehyde post-coated lipoplex (c) at a dose of 30 μg pDNA/animal afforded a transfection efficiency of about 10% of the positive adenoviral control. The other samples afforded no measurable transfection efficacy.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in biology, chemistry or related fields are intended to be within the scope of the following claims.

### REFERENCES

1. Zhu, J., Munn, R₁ J., and Nantz, M. H. (2000) Journal of the American Chemical Society 122, 2645-2646.
2. Rui, Y. J., Wang, S., Low, P. S., and Thompson, D. H. (1998) Journal of the American Chemical Society 120, 11213-11218.
3. Boomer, J. A., and Thompson, D. H. (1999) Chemistry and Physics of Lipids 99, 145-153.
4. Tang, F. X., and Hughes, J. A. (1998) Biochemical and Biophysical Research Communications 242, 141-145.
5. Tang, F. X., Wang, W., and Hughes, J. A. (1999) Journal of Liposome Research 9, 331-347.
6. Tang, F. X., and Hughes, J. A. (1999) Bioconjugate Chemistry 10, 791-796.
7. Byk, G., Wetzer, B., Frederic, M., Dubertret, C., Pitard, B., Jaslin, G., and Scherman, D. (2000) Journal of Medicinal Chemistry 43, 4377-4387.
8. Zalipsky, S., Qazen, M., Walker, J. A., Mullah, N., Quinn, Y. P., and Huang, S. K. (1999) Bioconjugate Chemistry 10, 703-707.
9. Blessing, T., Kursa, M., Holzhauser, R., Kircheis, R., and Wagner, E. (2001) Bioconjugate Chemistry 12, 529-537.
10. Xu, L. (2001) in United States patent application, George Town University, Washington D.C., USA.
11. Murray, K. D., Etheridge, C. J., Shah, S. I., Matthews, D. A., Russell, W., Gurling, H. M. D., and Miller, A. D. (2001) Gene Therapy 8, 453-460.
12. Kratz, F., Beyer, U. & Schüitte, M. T. Drug-Polymer Conjugates Containing Acid-Cleavable Bonds. Critical Reviews in Therapeutic Drug Carrier Systems 16, 245-287 (1999).
13. Franssen, E. J. F. et al. Low molecular weight proteins as carriers for renal drug targeting: Preparation of drug-protein conjugates and drug-spacer derivatives and their catabolism in renal cortex homogenates and lysosomal lysates. Journal of Medicinal Chemistry 35, 1246 (1992).
14. Lavie, E. et al. Monoclonal antibody L6-daunomycin conjugates constructed to release free drug at the lower pH of tumour tissue. Cancer Immunology Immunotherapy 33, 223 (1991).
15. Shen, W. T. & Ryser, H. J. P. Cis-aconityl spacer between daunomycin and macromolecular barriers: A model of pH-sensitive linkage releasing drug from a lysosomotropic conjugate. Biochemical and Biophysical Research Communications (1981).
16. Al-Shamkhani, A & Duncan, R₁ Sunthesis, controlled release properties and antitumour activity of alginatecis-aconityl-daunomycin conjugates. International Journal of Pharmaceutics 122, 107-119 (1995).

1. A delivery vehicle for a therapeutic agent comprising a modified lipid and a therapeutic agent;
wherein the modified lipid comprises a lipid and a delivery, targeting or stabilising moiety (DTS moiety);
wherein the lipid is linked to the DTS moiety via a linker which is stable in extracellular biological fluid and which is unstable in intracellular biological fluid and/or defined conditions; and
wherein the DTS moiety is linked to the lipid after formation of a complex of lipid and therapeutic agent.

2. A process for the preparation of delivery vehicle for a therapeutic agent comprising a modified lipid and a therapeutic agent, the process comprising the steps of;
(a) forming a complex of a lipid comprising a linker moiety and the therapeutic agent;
(b) linking a delivery, targeting or stabilising moiety (DTS moiety) to the lipid via the linker moiety, wherein the link between the DTS moiety and the lipid is stable in biological fluid and is unstable in defined conditions.

3. The vehicle according to claim 1 wherein the link is unstable on contact with a cell surface or within a cell.

4. The vehicle according to claim 1 wherein the link is unstable at defined pH conditions.

5. The vehicle according to claim 4 wherein the link is unstable at a pH of from 5.0 to 6.5.

6. The vehicle according to claim 1 wherein the link is unstable under reductive conditions or in intracellular biological fluid.

7. The vehicle according to claim 1 wherein the modified lipid is of the formula 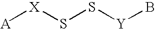
wherein one of A and B is a lipid and the other of A and B is a delivery, targeting or stabilising moiety (DTS moiety); wherein X and Y are independently optional linker groups.

8. The vehicle according to claim 1 wherein the modified lipid is of the formula 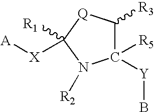
wherein one of A and B is a lipid and the other of A and B is a delivery, targeting or stabilising moiety (DTS moiety);
wherein X and Y are independently optional linker groups;
wherein R₁ is H or a hydrocarbyl group;
wherein R₂ is a lone pair or R₄, wherein R₄ is a suitable substituent;
wherein R₃ and R₅ are independently selected from H and a hydrocarbyl group; and
wherein Q is selected from O, S, NH.

9. The vehicle according to claim 1 wherein the modified lipid is of the formula 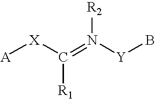
wherein one of A and B is a lipid and the other of A and B is a delivery, targeting or stabilising moiety (DTS moiety);
wherein X and Y are independently optional linker groups;
wherein R₁ is H, O⁻ or a hydrocarbyl group; and
wherein R₂ is a lone pair or R₄, wherein R₄ is a suitable substituent.

10. The vehicle according to claim 9 wherein the modified lipid is of the formula 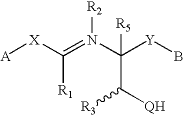
wherein one of A and B is a lipid and the other of A and B is a delivery, targeting or stabilising moiety (DTS moiety);
wherein X and Y are independently optional linker groups; and
wherein R₁ is H, O⁻ or a hydrocarbyl group.
wherein R₂ is a lone pair or R₄, wherein R₄ is a suitable substituent;
wherein R₃ and R₅ are independently selected from H and a hydrocarbyl group; and
Q is a suitable substituent.

11. The vehicle according to claim 8 wherein Q is selected from OH, SH, primary amines, secondary amines, tertiary amines and hydrocarbyl.

12. The vehicle according to claim 8 wherein R₁ is H.

13. The vehicle according to claim 8 wherein the C═N bond is acid labile or acid resistant.

14. The vehicle according to claim 13 wherein the C═N bond is acid labile.

15. The vehicle according to claim 13 wherein the C═N bond is acid resistant.

16. The vehicle according to claim 7 wherein Y is present.

17. The vehicle according to claim 16 wherein Y is O.

18. The vehicle according to claim 16 wherein Y is a hydrocarbyl group.

19. The vehicle according to claim 18
wherein Y is selected from —[CₙHₙ₋₂]ₐ—[NH]_{b}—[CZ]_{c}—[NH]_{d}—[CZ]ₑ—NH—
wherein a, b, c, d and e are independently selected from 0 to 10;
wherein n is from 5 to 10; and
wherein Z is O or S.

20. The according to claim 19 wherein a is 0 or 1.

21. The vehicle according to claim 19 wherein b is 0 or 1.

22. The vehicle according to claim wherein c is 0 or 1.

23. The vehicle according to claim 19 wherein d is 0, 1 or 2.

24. The vehicle according to claim 19 wherein e is 0 or 1.

25. The vehicle according to claim 19 wherein Z is O.

26. The vehicle according to claim 19 wherein n is 5.

27. The vehicle according to claim 16 wherein Y is selected from —NH—, —NH—CO—NH—, —NH—CS—NH—, —NH—CO—NH—NH—CO—NH—, —CO—NH—, and —C₅H₃—NH—, —NH—(CH₂)₂—NH—C(O)—CH(CH₂OH)—, —NH—(CH₂)₂—NH—C(O)—CH(CH₂SH)—, —NH—(CH₂)₂—NH—C(O)—CH₂O—, —NH—(CH₂)₂—NH—(CH₂)₃—NH—C(O)—CH(CH₂OH)—, —NH—(CH₂)₂—NH—(CH₂)₃—NH—C(O)—CH(CH₂SH)—, —NH—(CH₂)₂—NH—(CH₂)₃—NH—C(O)—CH₂O—, and —NH—CH₂—C(O)—NH—.

28. The vehicle according to claim 27 wherein Y is selected from —NH—(CH₂)₂—NH—C(O)—CH(CH₂OH)—, —NH—(CH₂)₂—NH—C(O)—CH(CH₂SH)—, —NH—(CH₂)₂—NH—C(O)—CH₂O—, —NH—(CH₂)₂—NH—(CH₂)₃—NH—C(O)—CH(CH₂OH)—, —NH—(CH₂)₂—NH—(CH₂)₃—NH—C(O)—CH(CH₂SH)—, —NH—(CH₂)₂—NH—(CH₂)₃—NH—C(O)—CH₂O—, —NH—CH₂—C(O)—NH—, and —NH—.

29. The vehicle according to claim 7 wherein X is present.

30. The vehicle according to claim 29 wherein X is a hydrocarbyl group.

31. The vehicle according to claim 7 wherein is a DTS moiety and B is a lipid.

32. The vehicle according to claim 1 wherein the DTS moiety is a delivery and/or stabilising moiety.

33. The vehicle according to claim 1 wherein the DTS moiety is a delivery and/or stabilising polymer.

34. The vehicle according to claim 1 wherein the DTS moiety is selected from mono or bifunctional poly(ethyleneglycol) (“PEG”), poly(vinyl alcohol) (“PVA”); other poly(alkylene oxides) such as poly(propylene glycol) (“PPG”); and poly(oxyethylated polyols) such as poly(oxyethylated glycerol), poly(oxyethylated sorbitol), and poly(oxyethylated glucose), and the like.

35. The vehicle according to claim 1 wherein the DTS moiety comprises a further linker group capable of linking to a further DTS moiety.

36. The vehicle according to claim 35 wherein the DTS moiety comprises a further linker group capable of linking to a targeting moiety.

37. The vehicle according to claim 1 wherein the lipid is or comprises a cholesterol group

38. The vehicle according to claim 37 wherein the cholesterol group is cholesterol.

39. The vehicle according to claim 37 wherein the cholesterol group is linked to X via a carbamoyl linkage or an ether linkage.

40. The vehicle according to claim 7 wherein the lipid is linked to X via a polyamine group.

41. The vehicle according to claim 40 wherein the polyamine group is not a naturally occurring polyamine.

42. The vehicle according to claim 40 wherein the polyamine group contains at least two amines of the polyamine group which are spaced from each other by an ethylene (—CH₂CH₂—) group.

43. The vehicle according to claim 42 wherein the polyamine is any one of spermidine, spermine or caldopentamine.

44. A modified lipid of the formula 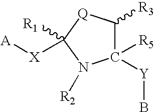
wherein one of A and B is a lipid and the other of A and B is a delivery, targeting or stabilising moiety (DTS moiety);
wherein X and Y are independently optional linker groups;
wherein R₁ is H or a hydrocarbyl group;
wherein R₂ is a lone pair or R₄, wherein R₄ is a suitable substituent;
wherein R₃ and R₅ are independently selected from H and a hydrocarbyl group; and
wherein Q is selected from OH, SH, NH.

45. A modified lipid of the formula 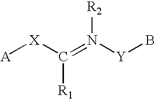
wherein one of A and B is a lipid and the other of A and B is a delivery, targeting or stabilising moiety (DTS moiety);
wherein X and Y are independently optional linker groups;
wherein R₁ is H, O⁻ or a hydrocarbyl group; and
wherein R₂ is a lone pair or R₄, wherein R₄ is a suitable substituent.

46. A modified lipid of the formula 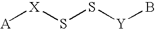
wherein one of A and B is a lipid and the other of A and B is a delivery, targeting or stabilising moiety (DTS moiety); wherein X and Y are independently optional linker groups.

47. (Cancel)

48. The compound of claim 44 in admixture with or associated with a nucleotide sequence or a pharmaceutically active agent.

49. A method for therapy comprised of employing the delivery vehicle according to claim 1.

50. A method for manufacture of a medicament for the treatment of a genetic disorder, condition, or disease, wherein a delivery vehicle according to claim 1 is employed.

51. A liposome/lipoplex formed from the compound of claim 44.

52. A method of preparing a liposome/lipoplex comprising forming the liposome/lipoplex from the compound according to claim 44.

53. A therapy comprised of administering an effective amount of the liposome/lipoplex according to claim 51.

54. A treatment of a genetic disorder or condition or disease employing a liposome/lipoplex according to claim 51.

55. A compound comprised of a nucleotide sequence and the delivery vehicle according to claim 1.

56. A compound according to claim 55 for use in therapy.

57. A medicament for the treatment of genetic disorder or condition or disease including the compound of claim 55.

58. A pharmaceutical composition comprising a delivery vehicle according to claim 1 admixed with a pharmaceutical and, optionally, admixed with a pharmaceutically acceptable diluent, carrier or excipient.
